(19) United States
(12) Patent Application Publication
(10) Pub. No.: US 2021/0391081 A1
(43) Pub. Date: Dec. 16, 2021

(54) PREDICTIVE GUIDANCE SYSTEMS FOR PERSONALIZED HEALTH AND SELF-CARE, AND ASSOCIATED METHODS

(71) Applicant: INFORMED DATA SYSTEMS INC. d/b/a ONE DROP, New York, NY (US)

(72) Inventors: Daniel R. Goldner, Minnetonka, MN (US); Ydo Wexler, Haifa (IL); Daniel Michael Alexander Barg, Astoria, NY (US); Jorge J. Guerra Marin, Houston, TX (US); Veena Samir Patel, Albuquerque, NM (US); Jeffrey Dachis, Brooklyn, NY (US)

(21) Appl. No.: 17/338,570

(22) Filed: Jun. 3, 2021

Related U.S. Application Data
(60) Provisional application No. 63/034,333, filed on Jun. 3, 2020.

(51) Int. Cl. G16H 50/20; G16H 10/60; G16H 50/30; G16H 50/70; A61B 5/00

(52) U.S. Cl. CPC G16H 50/20; G16H 10/60; G16H 20/60; G16H 50/70; A61B 5/7275; G16H 50/30

(57) ABSTRACT

Systems and methods for biomonitoring and personalized healthcare are disclosed herein. In some embodiments, a biomonitoring and healthcare guidance system is configured to generate personalized self-care recommendations (e.g., recommendations relating to sleep, exercise, diet, etc.) to guide a. patient in effectively managing and/or improving a chronic condition (e.g., diabetes, pre-diabetes, hypertension, hyperlipidemia, etc.). The system can continuously or periodically update and/or adapt the self-care recommendations (e.g., based on data from the particular patient as well as data from a plurality of other patients). The system can guide individuals toward self-care changes that are likely to improve their chronic health conditions, support them in making those changes, and adapt and/or update continuously over time.

### CROSS REFERENCE TO RELATED APPLICATION

This application claims priority to U.S. Provisional Patent Application No. 63/034,333, filed Jun. 3, 2020, the contents of which is incorporated herein by reference in its entirety.

### TECHNICAL FIELD

This disclosure relates generally to personalized healthcare and, in particular, to systems and methods for biomonitoring and healthcare guidance.

### BACKGROUND

Many individuals suffer from chronic health conditions, such as diabetes, pre-diabetes, hypertension, hyperlipidemia, and the like. For example, diabetes mellitus (DM) is a group of metabolic disorders characterized by high blood glucose levels over a prolonged period. Typical symptoms of such conditions include frequent urination, increased thirst, increased hunger, etc. If left untreated, diabetes can cause many complications. There are three main types of diabetes: Type 1 diabetes, Type 2 diabetes, and gestational diabetes. Type 1 diabetes results from the pancreas' failure to produce enough insulin. In Type 2 diabetes, cells fail to respond to insulin properly. Gestational diabetes occurs when pregnant women without a previous history of diabetes develop high blood glucose levels.

Diabetes affects a significant percentage of the world's population. Timely and proper diagnoses and treatment are essential to maintaining a relatively healthy lifestyle for individuals with diabetes. Application of treatment typically relies on accurate determination of glucose concentration in the blood of an individual at a present time and/or in the future. However, conventional health monitoring systems may be limited in availability or accessibility. Thus, there is a need for improved systems and methods for biomonitoring and/or providing personalized healthcare recommendations or information for the treatment of diabetes and other chronic conditions.

### BRIEF DESCRIPTION OF THE DRAWINGS

FIG. 1 is a schematic diagram illustrating an exemplary computing environment in which a healthcare guidance system operates, in accordance with embodiments of the present technology.

FIG. 2 is a block diagram illustrating a method for developing a biomonitoring and healthcare guidance system, in accordance with embodiments of the present technology.

FIG. 3 is a graph illustrating the median cumulative change of average blood sugar over the steps of an optimizer configured in accordance with embodiments of the present technology.

FIG. 4 is a graph illustrating the relative improvement compared to the original predictions at each step of the optimizer, when grouped by the initial blood glucose level configured in accordance with embodiments of the present technology.

FIG. 5 is a graph illustrating the size of improvements to forecast outcomes verses the initial blood glucose level configured in accordance with embodiments of the present technology.

FIG. 6 is a graph illustrating the median change over runs for three groups with different initial blood pressure levels configured in accordance with embodiments of the present technology.

FIG. 7 is a graph illustrating the mean percentage improvement at each blood pressure level compared to original outcome predictions configured in accordance with embodiments of the present technology.

FIG. 8 is a graph illustrating the percentage of samples for each size of improvement relative to the outcome models predictions for different initial blood pressure levels configured in accordance with embodiments of the present technology.

FIG. 9 is a flowchart illustrating a method for selecting a self-care mode, in accordance with embodiments of the present technology.

FIG. 10 is a graph illustrating a blood glucose adjustment for users without known diabetes configured in accordance with embodiments of the present technology.

FIG. 11 is a graph illustrating a blood glucose adjustment for users with and without known diabetes configured in accordance with embodiments of the present technology.

FIG. 12 is a graph illustrating systolic blood pressure and risk score in accordance with embodiments of the present technology.

FIG. 13 is a schematic block diagram of a computing system or device configured in accordance with embodiments of the present technology.

FIGS. 14 and 15 are schematic diagrams illustrating exemplary computing environments in which a healthcare guidance system operates, in accordance with embodiments of the present technology.

FIG. 16A-16C illustrate example prompts and reinforcements for self-care modes in accordance with embodiments of the present technology.

### DETAILED DESCRIPTION

The present technology generally relates to systems and methods for biomonitoring and providing personalized healthcare. In some embodiments, a healthcare guidance system is configured to generate personalized self-care recommendations (e.g., recommendations relating to sleep, exercise, diet, etc.) to guide a patient in effectively managing and/or improving a chronic condition (e.g., diabetes, pre-diabetes, hypertension, hyperlipidemia, etc.). The system can continuously or periodically update and/or adapt the self-care recommendations, e.g., based on data from the particular patient as well as data from a plurality of other patients. The system can guide individuals toward self-care changes that are likely to improve their chronic health conditions, support them in making those changes, and adapt and/or update continuously over time.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology.

### Systems for Biomonitoring and Healthcare Guidance

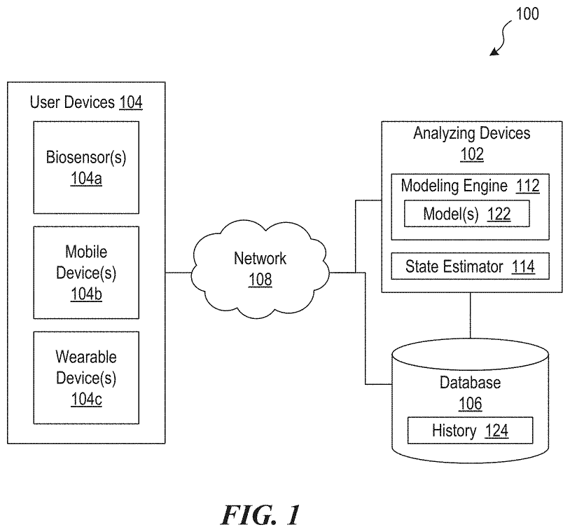

FIG. 1 is a schematic diagram of an exemplary computing environment in which a biomonitoring and healthcare guidance system 100 (“system 100”) operates, in accordance with embodiments of the present technology. As shown in FIG. 1, the system 100 can include one or more analyzing devices, one or more user devices 104, and/or at least one database or storage component 106 (“database 106”) operably coupled to each other, such as via a network 108. The system 100 is also operably coupled to at least one database or storage component 106 (“database 106”). The system 100 can include processors, memory, and/or other software and/or hardware components configured to implement the various methods described herein. For example, the system 100 can be configured to monitor a patient/user health state and provide predictive self-care guidance, as described in greater detail below.

For example, the user devices 104 can obtain biometric data, such as temperature, heartrate, blood pressure, blood glucose level or the like, and/or spatial data, such as location, acceleration, velocity, orientation, a change thereof over time, or the like. The user devices 104 can also obtain contextual data, such as user calendar (including, e.g., event name or category, location, date, time, participant, etc.), that may be used to categorize other obtained data. For example, the contextual data may be used to categorize the data into user activities or user health states.

The health state can be any status, condition, parameter, etc. that is associated with or otherwise related to the patient's health. In some embodiments, the system 100 can be used to identify, manage, monitor, and/or provide recommendations relating to diabetes, hypoglycemia, hyperglycemia, pre-diabetes, hypertension, hyperlipidemia, ketoacidosis, liver failure, congestive heart failure, hypoxia, kidney function, intoxication, dehydration, hyponatremia, shock, sepsis, trauma, water retention, bleeding, endocrine disorders, asthma, lung conditions, muscle breakdown, malnutrition, body function (e.g., lung functions, heart functions, etc.), physical performance (e.g., athletic performance), anaerobic activity, weight loss/gain, nutrition, wellness, mental health, focus, effects of medication, medication levels, health indicators, and/or user compliance. In some embodiments, the system 100 receives input data and performs monitoring, processing, analysis, forecasting, interpretation, etc. of the input data in order to generate instructions, notifications, recommendations, support, and/or other information to the patient that may be useful for self-care of diseases or conditions, such as chronic conditions (e.g., diabetes (type 1 and type 2), pre-diabetes, hypertension, hyperlipidemia, etc.).

The input data for the system 100 can include health-related information, contextual information, and/or any other information relevant to the patient's health state. For example, health-related information can include levels or concentrations of a biomarker, such as glucose, electrolytes, neurotransmitters, amino acids, hormones, alcohols, gases (e.g. oxygen, carbon dioxide, etc.), creatinine, blood urea nitrogen (BUN), lactic acid, drugs, pH, cell count, and/or other biomarkers. Health-related information can also include physiological and/or behavioral parameters, such as vitals (e.g., heart rate, body temperature (such as skin temperature), blood pressure (such as systolic and/or diastolic blood pressure), respiratory rate), cardiovascular data (e.g., pacemaker data, arrhythmia data), body function data, meal or nutrition data (e.g., number of meals; timing of meals; number of calories; amount of carbohydrates, fats, sugars, etc.), physical activity or exercise data (e.g., time and/or duration of activity; activity type such as walking, running, swimming; strenuousness of the activity such as low, moderate, high; etc.), sleep data (e.g., number of hours of sleep, average hours of sleep, variability of hours of sleep, sleep-wake cycle data, data related to sleep apnea events, sleep fragmentation (such as fraction of nighttime hours awake between sleep episodes, etc.), stress level data (e.g., cortisol and/or other chemical indicators of stress levels, perspiration), a1c data, etc. Health-related information can also include medical history data (e.g., weight, age, sleeping patterns, medical conditions, cholesterol levels, disease type, family history, patient health history, diagnoses, tobacco usage, alcohol usage, etc.), diagnostic data (e.g., molecular diagnostics, imaging), medication data (e.g., timing and/or dosages of medications such as insulin), personal data (e.g., name, gender, demographics, social network information, etc.), and/or any other data, and/or any combination thereof. Contextual information can include user location (e.g., GPS coordinates, elevation data), environmental conditions (e.g., air pressure, humidity, temperature, air quality, etc.), and/or combinations thereof.

In some embodiments, a guidance system or analyzing devices 102 (“analyzing device 102”) receive the input data from one or more user devices 104. The user devices 104 can be any device associated with a patient or other user, and can be used to obtain healthcare information, contextual information, and/or any other relevant information relating to the patient and/or any other users or patients (e.g., appropriately anonymized patient data). In the illustrated embodiment, for example, the user devices 104 can include at least one biosensor 104a (e.g., blood glucose sensors, pressure sensors, heart rate sensors, sleep trackers, temperature sensors, motion sensors, or other biomonitoring devices), at least one mobile device 104b (e.g., a smartphone or tablet computer), and, optionally, at least one wearable device 104c (e.g., a smartwatch, fitness tracker). In other embodiments, however, one or more of the devices 104a-c can be omitted and/or other types of user devices can be included, such as computing devices (e.g., personal computers, laptop computers, etc.). Additionally, although FIG. 1 illustrates the biosensor(s) 104a as being separate from the other user devices 104, in other embodiments the biosensor(s) 104a can be incorporated into another user device 104.

The biosensor 104a can include various types of sensors, such as chemical sensors, electrochemical sensors, optical sensors (e.g., optical enzymatic sensors, opto-chemical sensors, fluorescence-based sensors, etc.), spectrophotometric sensors, spectroscopic sensors, polarimetric sensors, calorimetric sensors, iontophoretic sensors, radiometric sensors, and the like, and combinations thereof. In some embodiments, the biosensor 104a is or includes a blood glucose sensor. The blood glucose sensor can be any device capable of obtaining blood glucose data from the patient, such as implanted sensors, non-implanted sensors, invasive sensors, minimally invasive sensors, non-invasive sensors, wearable sensors, etc. The blood glucose sensor can be configured to obtain samples from the patient (e.g., blood samples) and determine glucose levels in the sample. Any suitable technique for obtaining patient samples and/or determining glucose levels in the samples can be used. In some embodiments, for example, the blood glucose sensor can be configured to detect substances (e.g., a substance indicative of glucose levels), measure a concentration of glucose, and/or measure another substance indicative of the concentration of glucose. The blood glucose sensor can be configured to analyze, for example, body fluids (e.g., blood, interstitial fluid, sweat, etc.), tissue (e.g., optical characteristics of body structures, anatomical features, skin, or body fluids), and/or vitals (e.g., heat rate, blood pressure, etc.) to periodically or continuously obtain blood glucose data. Optionally, the blood glucose sensor can include other capabilities, such as processing, transmitting, receiving, and/or other computing capabilities. In some embodiments, the blood glucose sensor can include at least one continuous glucose monitoring (CGM) device or sensor that measures the patient's blood glucose level at predetermined time intervals. For example, the CGM device can obtain at least one blood glucose measurement every minute, 2 minutes, 5 minutes, 10 minutes, 15 minutes, 20 minutes, 30 minutes, 60 minutes, 2 hours, etc. In some embodiments, the time interval is within a range from 5 minutes to 10 minutes.

In some embodiments, some or all of the user devices 104 may be configured to continuously obtain any of the above data (e.g., health-related information and/or contextual information) from the patient over a particular time period (e.g., hours, days, weeks, months, years). For example, data can be obtained at a predetermined time interval (e.g., e.g., in the order of seconds, minutes, or hours), at random time intervals, or combinations thereof. The time interval for data collection can be set by the patient, by another user (e.g., a physician), by the analyzing devices 102, or by the user device 104 itself (e.g., as part of an automated data collection program). The user device 104 can obtain the data automatically or semi-automatically (e.g., by automatically prompting the patient to provide such data at a particular time), or from manual input by the patient (e.g., without prompts from the user device 104). The continuous data may be obtained by the system (e.g., collected at the analyzing devices 102) at predetermined time intervals (e.g., once every minute, 2 minutes, 5 minutes, 10 minutes, 15 minutes, 20 minutes, 30 minutes, 60 minutes, 2 hours, etc.), continuously, in real-time, upon receiving a query, manually, automatically (e.g., upon detection of new data), semi-automatically, etc. The time interval at which the user device 104 obtains data may or may not be the same as the time interval at which the user device 104 transmit the data to the analyzing devices 102.

The user devices 104 can obtain any of the above data and can provide output in various ways, such as using one or more of the following components: a microphone (either a separate microphone or a microphone imbedded in the device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, a sensor (e.g., a sensor included in or operably coupled to the user device 104), and/or any other device. The data obtained by the user devices 104 can include metadata, structured content data, unstructured content data, embedded data, nested data, hard disk data, memory card data, cellular telephone memory data, smartphone memory data, main memory images and/or data, forensic containers, zip files, files, memory images, and/or any other data/information. The data can be in various formats, such as text, numerical, alpha-numerical, hierarchically arranged data, table data, email messages, text files, video, audio, graphics, etc. Optionally, any of the above data can be filtered, smoothed, augmented, annotated, or otherwise processed (e.g., by the user devices 104 and/or the analyzing devices 102) before being used.

In some embodiments, any of the above data can be queried by one or more of the user devices 104 from one or more databases (e.g., the database 106, a third-party database, etc.). The user device 104 can generate a query and transmit the query to the analyzing devices 102, which can determine which database may contain requisite information and then connect with that database to execute a query and retrieve appropriate information. In other embodiments, the user device 104 can receive the data directly from the third-party database and transmit the received data to the analyzing devices 102, or can instruct the third-party database to transmit the data to the analyzing devices 102. In some embodiments, the analyzing devices 102 can include various application programming interfaces (APIs) and/or communication interfaces that can allow interfacing between user devices 104, databases, and/or any other components.

Optionally, the analyzing devices 102 can also obtain any of the above data from various third-party sources, e.g., with or without a query initiated by a user device 104. In some embodiments, the analyzing devices 102 can be communicatively coupled to various public and/or private databases that can store various information, such as census information, health statistics (e.g., appropriately anonymized), demographic information, population information, and/or any other information. Additionally, the analyzing devices 102 can also execute a query or other command to obtain data from the user devices 104 and/or access data stored in the database 106. The data can include data related to the particular patient and/or a plurality of patients or other users (e.g., health-related information, contextual information, etc.) as described herein.

The database 106 can be used to store various types of data obtained and/or used by the analyzing devices 102. For example, any of the above data can be stored as user history 124 in the database 106. The database 106 can also be used to store data generated by the system 100, such as previous predictions or forecasts produced by the system 100. In some embodiments, the database 106 includes data for multiple users, such as a plurality of patients (e.g., at least 50, 100, 200, 500, 1000, 2000, 3000, 4000, 5000, or 10,000 different patients). The data can be appropriately anonymized to ensure compliance with various privacy standards. The database 106 can store information in various formats, such as table format, column-row format, key-value format, etc. (e.g., each key can be indicative of various attributes associated with the user and each corresponding value can be indicative of the attribute's value (e.g., measurement, time, etc.)). In some embodiments, the database 106 can store a plurality of tables that can be accessed through queries generated by the analyzing devices 102 and/or the user devices 104. The tables can store different types of information (e.g., one table can store blood glucose measurement data, another table can store user health data, etc.), where one table can be updated as a result of an update to another table.

In some implementations, the system 100 can collect and analyze periodically (e.g., hourly, daily, weekly, monthly, etc.) the user health data. The system 100 can identify (e.g., forecast) a health event (e.g., high or low blood pressure, high or low blood glucose levels, risk of cardiovascular disease, etc.) of the user based on the health data. The system 100 can select (or generate) a self-care mode with an action of sequence of actions (e.g., exercise, diet, sleep, reduce stress, etc.) for the user to perform to mitigate the risk of the health event or avoid the health event. The user can receive a notification (e.g., SMS message, email, alert on a user interface, etc.) which includes the forecasted health event, the actions to perform to mitigate or avoid the health event, and the self-care mode. For example, the system 100 can determine that the blood pressure of the user is too high and forecast that the user can experience a heart disease or heart disease if no user action is taken to lower the blood pressure. The system 100 can select a self-care mode which contains an action or sequence of actions directed towards lowering the blood pressure of the user. The user can receive a notification alerting them of their high blood pressure, the forecasted heart disease, and actions for the user to perform to avoid the heart disease. In some cases, the notification can include a recommendation to seek medical assistance based on the health data.

In some embodiments, one or more users can access the system 100 via the user devices 104, e.g., to send data to the analyzing devices 102 (e.g., health-related information, contextual information) and/or receive data from the system 100 (e.g., predictions, notifications, recommendations, instructions, support, etc.). The users can be individual users (e.g., patients, healthcare professionals, etc.), computing devices, software applications, objects, functions, and/or any other types of users and/or any combination thereof. For example, upon obtaining any of the input data discussed above, the user device 104 can generate an instruction and/or command to the analyzing devices 102, e.g., to process the obtained data, store the data in the database 106, extract additional data from one or more databases, and/or perform analysis of the data. The instruction/command can be in a form of a query, a function call, and/or any other type of instruction/command. In some implementations, the instructions/commands can be provided using a microphone (either a separate microphone or a microphone imbedded in the user device 104), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The user device 104 can also instruct the analyzing devices 102 to perform an analysis of data stored in the database 106 and/or inputted via the user device 104.

As discussed further below, the analyzing devices 102 can analyze the obtained input data, including historical data, current real-time data, continuously supplied data, and/or any other data (e.g., using a statistical analysis, machine learning analysis, etc.), and generate output data. The output data can include predictions of a patient's health state, interpretations, recommendations, notifications, instructions, support, and/or other information related to the obtained input data. The analyzing devices 102 can perform such analyses at any suitable frequency and/or any suitable number of times (e.g., once, multiple times, on a continuous basis, etc.). For example, when updated input data is supplied to the analyzing devices 102 (e.g., from the user devices 104), the analyzing devices 102 can reassess and update its previous output data, if appropriate. In performing its analysis, the analyzing devices 102 can also generate additional queries to obtain further information (e.g., from the user devices 104, the database 106, or third-party sources). In some embodiments, the user device 104 can automatically supply the analyzing devices 102 with such information. Receipt of updated/additional information can automatically trigger the analyzing devices 102 to execute a process for reanalyzing, reassessing, or otherwise updating previous output data.

In some embodiments, the analyzing devices 102 is configured to analyze the input data and generate the output data using one or more machine learning models 122. The machine learning models 122 can include supervised learning models, unsupervised learning models, semi-supervised learning models, and/or reinforcement learning models generated by one or more modeling engines 112. Examples of machine learning models suitable for use with the present technology include, but are not limited to: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning, support vector machines), regularization algorithms (e.g., ridge regression, least absolute shrinkage and selection operator, elastic net, least-angle regression), decision tree algorithms (e.g., classification and regression trees, Iterative Dichotomiser 3 (ID3), C4.5, C5.0, chi-squared automatic interaction detection, decision stump, M5, conditional decision trees), Bayesian algorithms (e.g., naïve Bayes, Gaussian naïve Bayes, multinomial naïve Bayes, averaged one-dependence estimators, Bayesian belief networks, Bayesian networks), clustering algorithms (e.g., k-means, k-medians, expectation maximization, hierarchical clustering), association rule learning algorithms (e.g., apriori algorithm, ECLAT algorithm), artificial neural networks (e.g., perceptron, multilayer perceptrons, back-propagation, stochastic gradient descent, Hopfield networks, radial basis function networks), deep learning algorithms (e.g., convolutional neural networks, recurrent neural networks, long short-term memory networks, stacked auto-encoders, deep Boltzmann machines, deep belief networks), dimensionality reduction algorithms (e.g., principle component analysis, principle component regression, partial least squares regression, Sammon mapping, multidimensional scaling, projection pursuit, discriminant analysis), time series forecasting algorithms (e.g., exponential smoothing, autoregressive models, autoregressive with exogenous input (ARX) models, autoregressive moving average (ARMA) models, autoregressive moving average with exogenous inputs (ARMAX) models, autoregressive integrated moving average (ARIMA) models, autoregressive conditional heteroskedasticity (ARCH) models), and ensemble algorithms (e.g., boosting, bootstrapped aggregation, AdaBoost, blending, stacking, gradient boosting machines, gradient boosted trees, random forest).

Although FIG. 1 illustrates a single set of user devices 104, it will be appreciated that the analyzing devices 102 can be operably and communicably coupled to multiple sets of user devices, each set being associated with a particular patient or user. Accordingly, the system 100 can be configured to receive and analyze data from a large number of patients (e.g., at least 50, 100, 200, 500, 1000, 2000, 3000, 4000, 5000, or any number of different patients) over an extended time period (e.g., weeks, months, years). The data from these patients can be used to train and/or refine one or more machine learning models implemented by the analyzing devices 102, as described below.

The analyzing devices 102 and user devices 104 can be operably and communicatively coupled to each other via the network 108. The network 108 can be or include one or more communications networks, and can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof. Additionally, although FIG. 1 illustrates the analyzing devices 102 as being directly connected to the database 106 without the network 108, in other embodiments the analyzing devices 102 can be indirectly connected to the database 106 via the network 108. Moreover, in other embodiments one or more of the user devices 104 can be configured to communicate directly with the analyzing devices 102 and/or database 106, rather than communicating with these components via the network 108.

The various components 102-108 illustrated in FIG. 1 can include any suitable combination of hardware and/or software. In some embodiment, components 102-108 can be disposed on one or more computing devices, such as, server(s), database(s), personal computer(s), laptop(s), cellular telephone(s), smartphone(s), tablet computer(s), and/or any other computing devices and/or any combination thereof. In some embodiments, the components 102-108 can be disposed on a single computing device and/or can be part of a single communications network. Alternatively, the components can be located on distinct and separate computing devices. For example, although FIG. 1 illustrates the analyzing devices 102 as being a single component, in other embodiments the analyzing devices 102 can be implemented across a plurality of different hardware components at different locations.

In some embodiments, the analyzing devices 102 may include a state estimator 114 configured to estimate a user context or a user health state. The state estimator 114 can use the above-described input data to determine a current or ongoing activity or state of the user. Similarly, the state estimator 114 can analyze the above-described input data to predict a future activity or health state of the user. The state estimator 114 can use corresponding machine learning models 122 to generate the estimated user states. For example, the state estimator 114 can use the models 122 to predict based on the current state and the user history 124 that blood glucose levels will reach a threshold level at a time when the user will likely be incapacitated (e.g., sleeping or intoxicated). In some embodiments, the state estimator 114 can generate activity predictions for a predetermined future duration based on the obtained data. The state estimator 114 can start from a current health state (e.g., blood glucose level) and extrapolate or derive future health states according to the activity predictions. The system 100 can use the future health states to determine and recommend a set of user actions that may be implemented between now and a future time to avoid the thresholding health states and/or to conform to a targeted behavior (e.g., a repeated set of user actions).

### Methods for Biomonitoring and Healthcare Guidance

In some embodiments, the healthcare guidance systems described herein (e.g., the system 100 of FIG. 1) are configured to generate personalized self-care recommendations/modes (e.g., recommendations relating to sleep, exercise, diet, or any other health-related action that can be performed by the patient without aid of a healthcare professional) to guide a patient in effectively managing and/or improving a chronic condition (e.g., diabetes, pre-diabetes, hypertension, hyperlipidemia, etc.). The system can continuously update and/or adapt the self-care recommendations, e.g., based on continuously-collected data from the particular patient as well as data from a plurality of other patients. The system can guide individuals toward self-care changes that are likely to improve their chronic health conditions, support them in making those changes, and adapt and/or update continuously over time.

In some embodiments, the systems described herein are configured to generate personalized self-care recommendations for a patient by performing the following processes: (1) prediction, (2) evaluation, (3) support, (4) monitoring, and/or (5) adaptation. These processes can be performed simultaneously and/or sequentially with each other. Each of these processes is described in detail below.

The prediction process can include using available information (e.g., health-related information, contextual information, etc.) as inputs to a predictive model that forecasts a person's health outcomes (“Outcomes Model”). The health outcomes can include biometric measures associated with health, such as average blood glucose concentration, average blood pressure, cholesterol levels, weight, heart health metrics, kidney function metrics, etc.

The evaluation process can include considering many modes of self-care (e.g., diet, physical activity, consistency of medication use, self-monitoring of behaviors or health metrics, sleep, tobacco use, alcohol use, meditation, etc.). The Outcomes Model can be used in combination with an optimization algorithm (“Optimizer”) to determine an improvement in outcome (relative to a current forecast of the patient's outcome) that may be achieved by improving and/or changing various modes of self-care. This information can be used to determine the mode of self-care that, at the current time, is likely to provide the best long-term improvement in outcomes.

The support process can include supporting the user (e.g., the patient) in making improvements in the self-care mode(s) identified in the evaluation process. Support for behavior changes can be provided in various ways, including coaching, education, peer support, and automated prompts, rewards, and/or feedbacks. In some embodiments, the system generates automated prompts, rewards, and/or feedbacks that are continuously adjusted and optimized separately for each individual each day through a model created for that purpose (“Adaptive Support Model”).

The monitoring process can include continuously collecting information about each user's program engagement, behaviors, biometrics, and/or outcomes. Some examples of the user's program engagement can include application use, interactions with coaches, logging, use of educational resources such as lesson completion, interactions with peer support networks, and others. Some examples of the collected behavior information can include physical activity, sleep, food and mealtimes, consistency of medication use, location, and the like. Some examples of the collected biometrics can include heart rate, blood pressure, skin temperature, blood glucose and other subcutaneous analytes, and others. Some examples of the collected outcomes can include: statistics of blood glucose concentration such as time in range, HbA1c, or average blood glucose; statistics of blood pressure including average and variability; statistics of cholesterol levels; other health statistics including weight, BMI, waist circumference and others; statistics of health markers for conditions including many cancers, heart disease, or others.

Data can be collected from user devices, including any of the following: wearable devices such as a biosensor, Apple watch, FitBit; from connected devices such as Bluetooth-enabled blood glucose meters, scales, or blood pressure cuffs; from integrations with other data collectors such as fitness and health apps; from user logging, which can be done through a mobile application or through an integration with a voice-controlled personal assistant such as Apple Sir or Amazon Alexa; or any other means. The collected data can be stored in a database with appropriate privacy and security protections. The system (e.g., biomonitoring and healthcare guidance system) for collecting and storing this information can be referred to herein as the “Monitoring System.” In daily use for the patient, information from that patient can be used as inputs to the Outcomes Model, Optimizer, and Adaptive Support Model. Also, information collected from all users of the Monitoring System (e.g., system 100) can be used periodically to train or retrain the Outcomes Model, Optimizer, and/or Adaptive Support Model.

The adaptation process can include repeating one or more of the prediction, evaluation, support, and/or monitoring processes discussed above to update and/or modify the self-care recommendations for the patient. One or more of these processes can be continually performed, such that the adaptation process is ongoing and responds in real-time to the patient's actions. For example, if the system recommends that the patient exercise, the system can quickly adapt based on the patient's subsequent actions. If no exercise takes place, the system can use the new information that exercise is not likely to happen at the moment, and recalculate to develop a new estimate of what mode of self-care will be most effective (e.g., a mode that is more likely to be used). If exercise does take place, the monitoring activity can provide data suggesting how much exercise is happening and/or how often, and that information can be used to determine whether the focus of support should stay with exercise, or move on to other modes (e.g., diet or sleep).

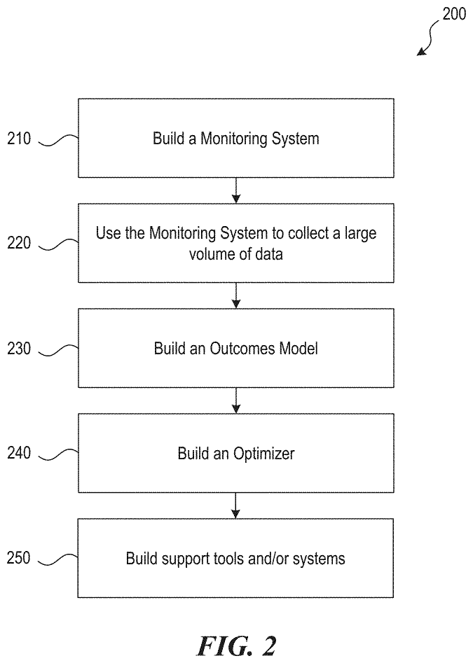

FIG. 2 is a block diagram illustrating a method 200 for developing a healthcare guidance system, in accordance with embodiments of the present technology. The method 200 begins at step 210 with building a Monitoring System (e.g., system 100). The Monitoring System can include a software application that is usable with user devices including advanced support for logging information, including mobile devices (e.g., smartphones) and/or wearable devices (e.g., smartwatches such as the Apple Watch). The Monitoring System can be configured for direct integration with other technology platforms and devices, such as Sir, Alexa, Fitbit, Dexcom, Apple Health, Google Fit, and/or other health, wellness, and fitness apps. The Monitoring System can also be integrated with connected biosensors and devices, such as glucometers, scales, blood pressure cuffs, wearable patches, etc. In building the Monitoring System, the various devices and/or platforms can be connected, such as via a registration process, a software, a common communication protocol, and/or a mobile application.

At step 220, the Monitoring System is used to collect a large volume of data. For example, the Monitoring System can collect over 24 billion data points from millions of users in all 195 countries. The Monitoring System can be used to collect various different types of data from the connected devices as described above. The collected data can be processed (e.g., grouped or combined via a predetermined mechanism) and stored in the system 100 for subsequent use.

At step 230, an Outcomes Model is built. Techniques for building the Outcomes Model are described in U.S. Provisional Patent Application No. 62/970,282 and U.S. patent application Ser. No. 16/888,105, both of which are incorporated by reference herein in their entireties.

At step 240, an Optimizer is built. In some embodiments, the purpose of the optimizer is to compute which self-care mode, if improved, will give the greatest improvement in outcomes. In other words, the Optimizer is intended to optimize the Outcomes Model. Various methods for optimizing a model (e.g., determining which inputs give the most desirable output, such as the best self-care mode) can be used. The self-care modes can include an activity, exercise, sleep, weight loss, medication, food, and/or meditation.

All behavior and signals can have a multitude of features that go into the outcome models. The Monitoring System can cluster the multitude of features by various categories such as based on A) the features are interdependent and cannot be changed without affecting other features, B) reducing the state space of possible modes that can be changed, or C) specific coherent self-care modes that the user improve in, where those related features reflect the overall changes that occur in user's behavior in that field.

To cluster the features, the Monitoring System can compute a distance matrix based on the variation of information of the features. For a pair of features f₁, f₂, the variation of information can be computed via VI(f₁, f₂)=H(f₁)+H(f₂)−2I(f₁, f₂), where H(f) is the entropy of f and I(f₁, f₂) is the mutual entropy between f₁ and f₂. Other distance metrics can be used to determine the distance between features. The features are clustered based on the distances computed. A method can be used known as Agglomerative Clustering, that iteratively creates hierarchical clusters based on a predetermined criterion for distance between sub-clusters. As with the distance metric, also here other clustering methods can be used.

A covariance matrix of features can be computed for each of the clusters. The Monitoring System use an implied conditional independence and normality assumption within each cluster to extract a change in a feature Y given a change of r_{X} in another from feature X: r_{Y}=r_{X}·C_{YX}C_{XX}⁻¹, where C is the covariance matrix for a cluster and C_{YX} is the corresponding entry for features X and Y. Modelling the changes of related features with less conditional independencies implied is also possible, and can require a more computationally involved inference. The Monitoring System can select a single feature from each cluster, preferably one feature that contributes the most to the predictions of the outcome model. This can be established via sensitivity analysis and measuring contribution via Shapley values or similar common estimation methods.

Two optimizing systems, Optimizer A and Optimizer B, described herein can rely on pre-processing and calculations. A feature or a cluster can refer to a set of features affected by change in a specific self-care mode. The Monitoring System can establish a step size for each cluster. In some cases, Monitoring System can establish a step size for each cluster based on one or more or all self-care mode having limits to the expected magnitude of change. Setting the step size stems from the possible changes in behavior taking from the dataset for multiple users (e.g., taking the 25th percentile from all non-zero changes in each feature, to determine, in the entire dataset over non-overlapping, fixed one-month periods). For a specific user the actual change considered can be the minimum between this global step size for the cluster and a set percentage from the feature's value, or another logic that is reasonable vis-a-vis the individual user's data.

Optimizer A (e.g., a Greedy Optimizer executing a Greedy Algorithm) can operate to propose an improvement in a self-care mode that will bring the largest expected change in outcomes, above and beyond that predicted by the outcomes model with no changes made to self-care. For example, at any moment for any individual, the algorithm can perform the following: A) use the Outcomes Model to make an outcomes forecast using the most recent available data for the individual; B) for each cluster representing a self-care mode, change the values of the representative feature, followed by the corresponding change in all other features in the cluster (e.g., reduce logged carbohydrate values for the most recent 30 days by the 5%, or increase recorded physical activity over the past 30 days by 10%) thus, using the covariance matrices for the clusters, re-create the input features for the Outcomes Model hypothetically improved data values, and recalculate the forecast from the revised inputs; C) analyze the revised outcomes forecasts to determine which change or changes produce the greatest improvement in the forecast. These change(s) can be selected as the recommended self-care actions for focus for that individual at that time.

In an example, the Monitoring System executed the Optimizer A for one-month predictions of average blood glucose for over 11,000 users and over 22,000 non-overlapping samples. The clusters included features related to: heart rate, blood pressure, physical activity, carbohydrate intake, sugar consumption, saturated fat consumption, sodium consumption, Vitamin C consumption, and weight related features. Other features were not clustered, and were not in the action space (i.e., not considered as possible self-care changes). The average improvement was recorded from following the decisions of the Optimizer A, relative to the predicted change from the Outcome Models.

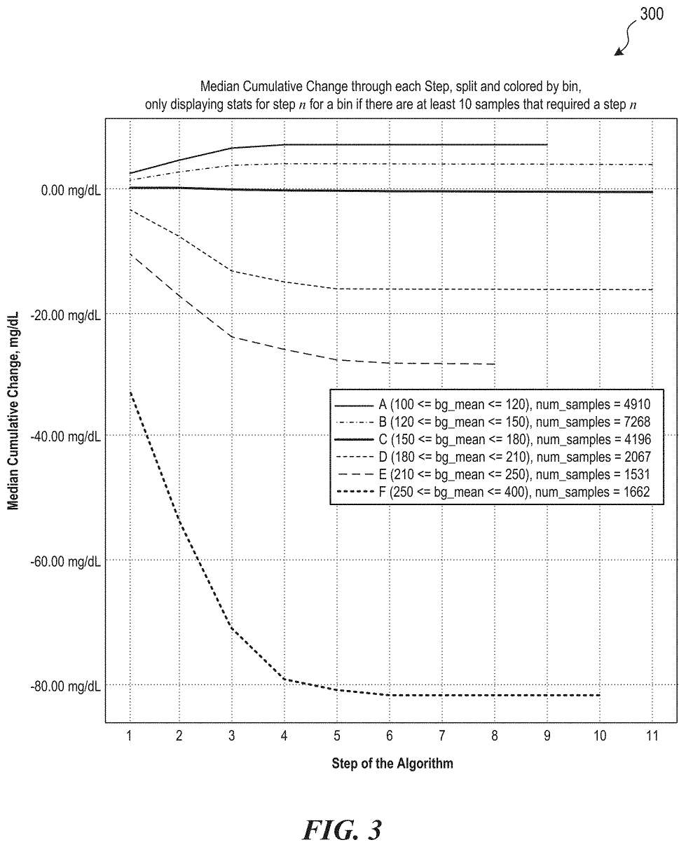

Blood glucose predictions can be illustrated as the cumulative change in predicted outcome over the steps of the steps of the optimizer. Example 300 in FIG. 3 illustrates a median cumulative change of average blood sugar over the steps of the optimizer A, for groups with different initial blood glucose levels. FIG. 3 illustrates the median change over runs for six groups with different initial blood glucose levels. As shown in FIG. 3, users with high initial blood glucose levels (over 180 mg/dL) tend to benefit the most, while users whose average blood glucose level is in the normal range don't achieve such benefits overall.

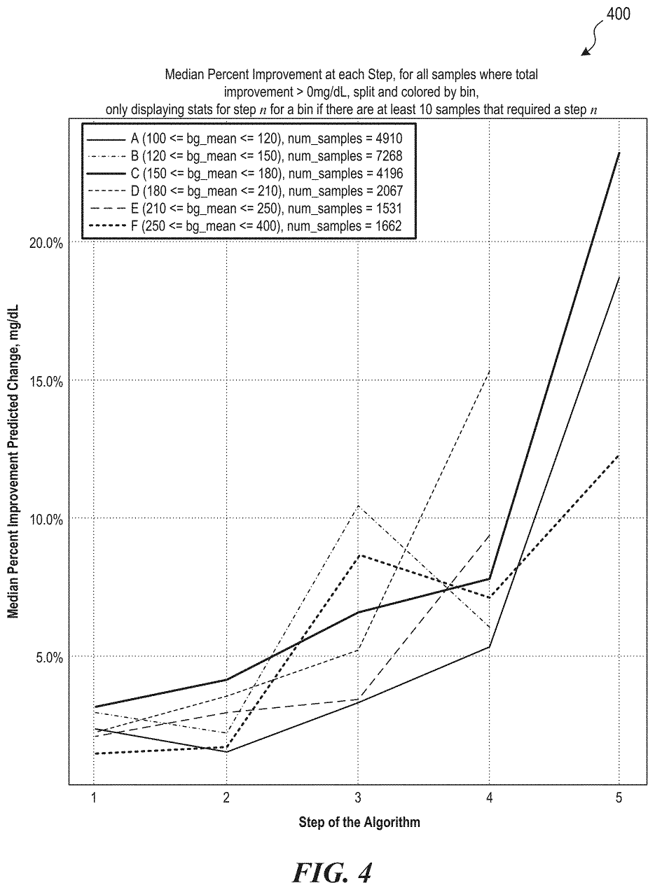

In an example, the improvement that the optimizer achieves over the original (non-intervention) outcome predictions was analyzed. Example 400 in FIG. 4 illustrates a median percentage improvement at each level compared to original outcome predictions without intervention. FIG. 4 illustrates the relative improvement compared to the original predictions at each step of the optimizer, when grouped by the initial blood glucose level. Even though FIG. 3 demonstrates a slight increase in blood glucose over time for groups with low starting blood glucose levels, FIG. 4 illustrates that following the interventions from the optimizers still decreases that verses the original predictions.

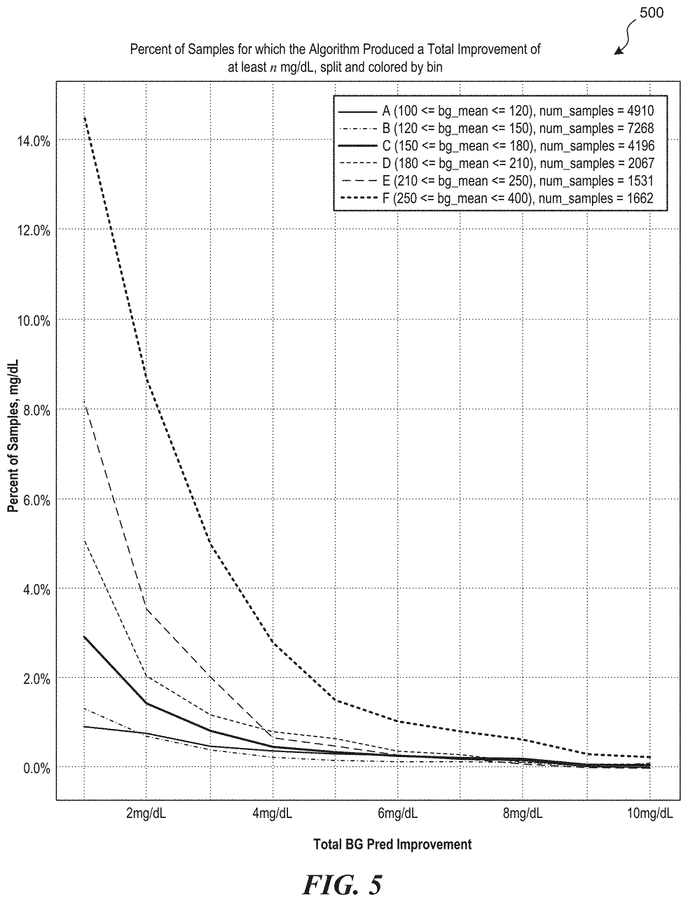

In an example, the size of improvements to forecast outcomes verses the initial blood glucose level was analyzed. Example 500 in FIG. 5 illustrates percentage of samples for each size of improvement relative to the Outcome Models predictions for different initial blood glucose levels. FIG. 5 illustrates the largest relative improvements in a small number of samples, most of them for users whose starting blood glucose level was high. Clusters that had to do with heart rate and blood pressure were the most common to be selected, and often were the selection of the algorithm in the first iteration.

In an example, similarly to improving blood glucose, the Monitoring System executed the Optimizer A for one-month predictions of average blood pressure for over 1,500 users and over 3,000 non-overlapping samples. The clusters included features related to behaviors and physical signals (e.g., features related to behaviors and physical signals of the blood glucose intervention as illustrated and described in FIGS. 3-5).

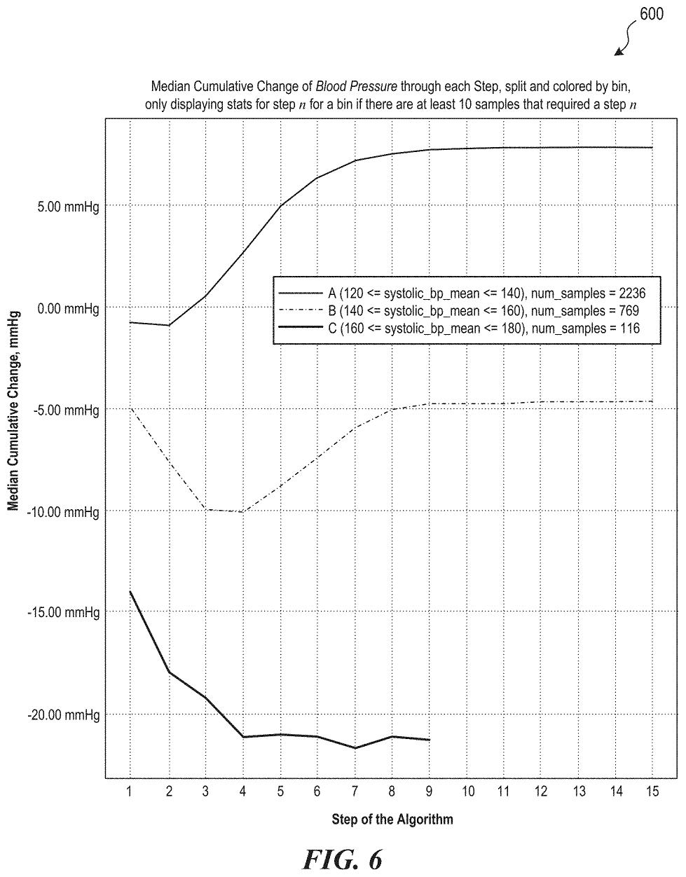

Example 600 in FIG. 6 illustrates a median cumulative change of average systolic blood pressure over the steps of the Optimizer A, for groups with different initial blood pressure levels (e.g., the cumulative change in predicted outcome over the steps of the optimization). FIG. 6 illustrates the median change over runs for three groups with different initial blood pressure levels. As evident from FIG. 6, users with high initial blood pressure levels (over 160 mmHg) tend to benefit the most, while users whose average blood pressure level is lower don't achieve such benefits overall. It is noted that many samples in group A, whose initial blood pressure levels were only moderately above normal, experience an increase in blood pressure after a few steps. Techniques for evaluating blood pressure systems are described in U.S. Provisional Patent Application No. 63/188,641 which is incorporated by reference herein in its entireties.

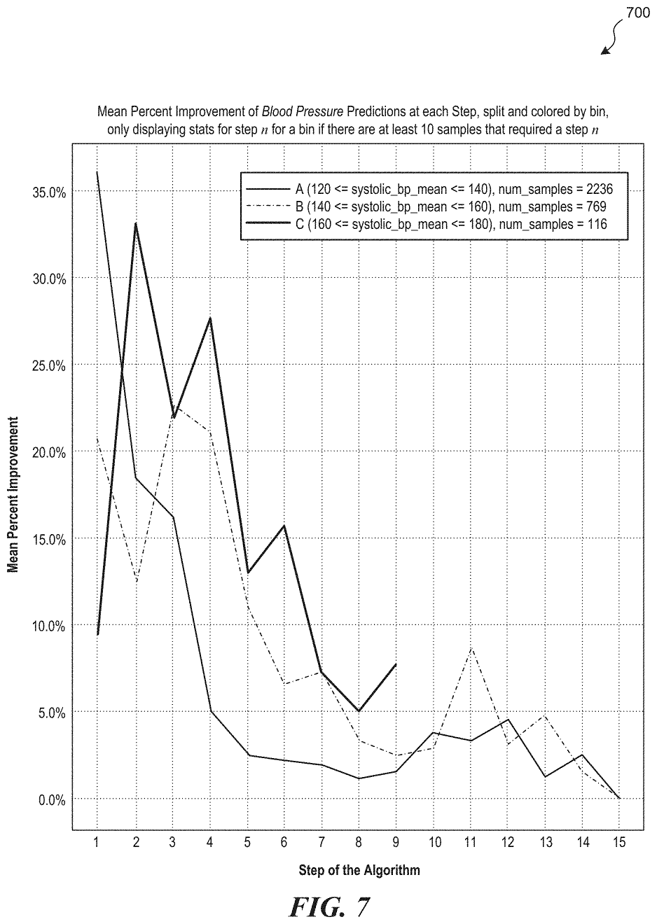

In an example, the Monitoring System recorded the average improvement from following the decisions of the greedy optimizer, relative to the predicted change from the Outcome Models. Example 700 in FIG. 7 illustrates the mean percentage improvement at each blood pressure level compared to original outcome predictions without intervention. Even though, as mentioned above, groups with low starting blood pressure levels show a slight increase in blood pressure over time (e.g., FIG. 6), FIG. 7 illustrates that following the interventions from the optimizers still decreases the blood pressure relative to the original rising predictions. In other words, the interventions delay or reduce the rise in blood pressure that was otherwise predicted.

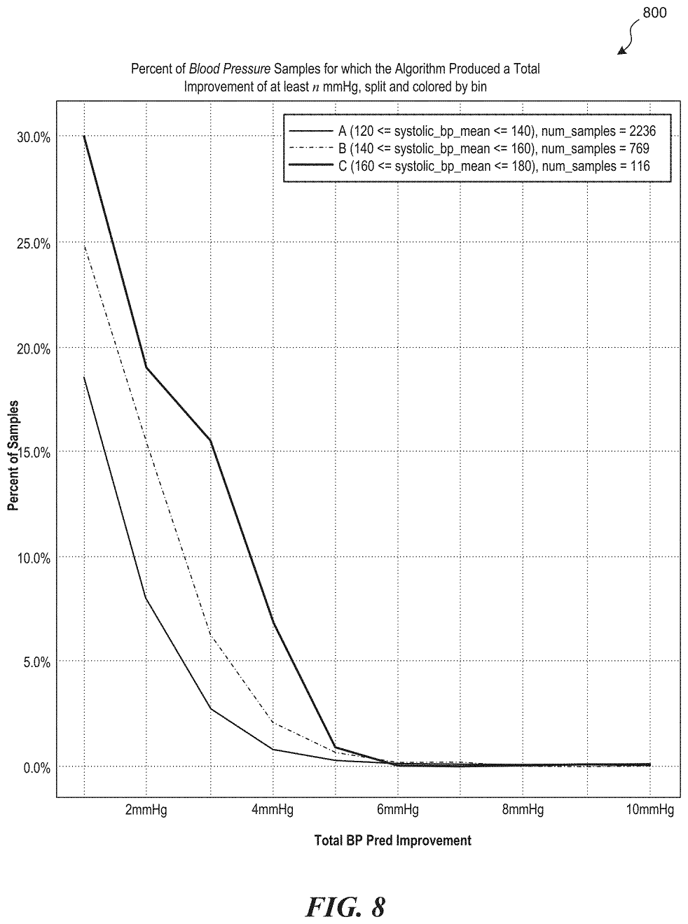

In an example, the Monitoring System can analyze how the size of improvements varied with the initial blood pressure level. Example 800 in FIG. 8 illustrates a percentage of samples for each size of improvement relative to the Outcome Models predictions for different initial blood pressure levels. Overall larger relative improvements can be achieved for samples where the initial blood pressure level is high. The largest relative improvements are seen in a small number of samples, most of them for users whose starting blood pressure level was high. Clusters that had to do with blood glucose, physical activity, and carbohydrate consumption were the most common to be selected, and often were the selection of the algorithm in the first iteration.

Optimizer B can operate to optimize for a longer set of interventions, taking into account the expected effect along the path of interventions (e.g., an optimization of expected discounted values can account for changes and interventions being performed in sequence). While, for a particular person at a particular time, intervention A (e.g., reducing carbohydrates) may give the greatest benefit of any single change, a greater overall benefit may be achieved by first performing intervention B (e.g., improving sleep consistency) and then doing intervention A. Optimizer B can maximize the expected value of an intervention, given the assumption that additional, optimal interventions may follow.

Overall, this system maximizes the discounted expected value along a path of interventions, where the term “discounted” is used because, in some embodiments, expected results are evaluated in such a way that there is a trade-off between achieving a better outcome (e.g. lower blood glucose concentration, lower blood pressure, lower weight, or improved kidney function, improved heart function, etc.) verses how long it takes to achieve. (e.g., a 10 mg/dL reduction in 4 months is preferable to an 11 mg/dL reduction that takes 8 years.)

The Monitoring System can use several methods for optimizing a sequence of inputs to a model in this way. In some implementations, the Monitoring System can use a method called Deep Q-learning (commonly called DQN, or Deep Q-Network), where an optimal policy is learned by a deep neural network model. The overall method of RL via Q-learning, is the approach of maximizing the expected sum of decaying rewards via the following formula: Q(s, α)=r(s, α)+γ−max_{α}, Q(s′,α′) for a state s and action a, where γ is a decay (or discount) factor, r(s, α) is the immediate reward that may be collected from performing action a when at state s (and in our case the predicted change in outcomes), and s′ is the new state to which the environment transitions to. States in this context are the values of features in the clusters, where only the representative feature from each cluster is represented in a state. The actions are similar to the ones enabled for the Optimizer A, and consist of a change to a single cluster, or self-care mode, by a step size that is determined as explained above. In the context of self-care guidance, a DQN-RL model learns a policy that maps from a state, which is the current values of representative features for a user, into estimated Q-values, one for each possible action, where the rewards are predicted changes in outcomes from the Outcome Models.

In some embodiments, the steps for making a decision using such reinforcement learning system with DQN are: 1) observe the current state of the user (from representative features' values); 2) get the deep-network's assessment of the Q-values for the different actions; 3) select the action that yields the best value. Such selection can be done stochastically with respect to the relative estimated Q-values. In some embodiments, For the model to keep learning the best action over time, and to tie it with the predictions of the Outcome Models, two more steps can be required: 1) record as immediate reward the change in outcome from the last decision time; and 2) add an example for the network to learn from, with the previous state as the input, the estimated Q-values as the output for all actions that were not selected, and Q=r+γ·Qₑ(s, α) where r is the observed reward, α the action that corresponds to the intervention taken in previous step, and Qₑ(s,α) is the estimated Q-value for the selected action.

The discounted expected value approach can use many different techniques for optimizing a sequence of inputs to a model. One approach is reinforcement learning. By defining an appropriate so-called action space (e.g., the list of possible changes to make) and an appropriate reward function (e.g., an improvement in outcome, multiplied by a factor that discounts improvements more if they are further away in time), a model is devised that computes the expected discounted reward for each possible action. The action with the greatest expected discounted reward can then be selected as the recommended action at the current time. With either Optimizer A or Optimizer B, or with any optimizer, the results are not limited only to the best choice. A ranked list of interventions can be produced, in order of decreasing expected effect on the outcomes, so that if the best mode is impractical to address for some reason, the second-best mode can be selected, and so on.

In one example using a simple sensitivity optimization as described above, baseline forecasts were made for a sample of users of a mobile app. Data on blood pressure, heart rate, physical activity, carbohydrates, and medications from the previous 30 days were systematically varied (e.g., increased or reduced by 10%). For each user, the change that led to the greatest improvement (e.g., reduction) in a 4-month forecast of the 30-day average blood glucose concentration was determined. The most effective change varied across individuals. For example, the following numbers of individuals had the following changes identified as the single most effective intervention:
Changing blood pressure: 1143 individuals
Changing heart rate: 631 individuals
Changing amount of physical activity: 366 individuals
Changing medication dosage or adherence: 143 individuals
Changing intake of carbohydrates: 57 individuals

Some of these changes, such as medication doses, can be evaluated by a medical professional, who can then recommend the change or a variation. Others, such as physical activity or carbohydrate intake, can be used to determine the main focus of an individual intervention guided by a human coach or an automated program delivered digitally. Still others, such as heart rate and blood pressure, may not able to be immediately, directly changed by the person, but can still indicate the best intervention of focus. For example, knowing that a lower heart rate is the best current driver of results can lead to a decision to focus on heart conditioning exercise, stress reduction and relaxation techniques, or other influencers of heart rate.

Referring back to FIG. 2, at step 250, tools and/or systems for supporting individuals in making recommended changes are built. The support tools and/or systems can include, for example, a digital platform to support individuals in self-care. The digital platform can include an application having facilities for logging and aggregating data, for working one-on-one through the application with a trained coach (e.g., a certified diabetes educator, dietician, nurse, psychologist, etc.), for accessing educational material related to self-care for the user's chronic condition, and/or for interacting with peers who are also managing chronic conditions. These capabilities can be activated and highlighted specifically for a given user based on the self-care mode recommended by the Optimizer as the best for improvement in that user's outcomes.

In some embodiments, recommendations can be provided to the user's coach, who can then explain the importance of the recommendation to the user, and support the user to follow a program emphasizing the recommended mode of self-care. Alternatively or in combination, the system can use the recommendation from the Optimizer to select an automated program designed to promote a particular behavior. For example, if the optimal self-care intervention for a particular user is to work on reducing blood pressure, an Adaptive Support Model can be established for the user, designed to improve consistency of self-monitoring of blood pressure and/or of any other habit related to reduction of blood pressure.

Once the system has been built as described above with respect to steps 210-250, the system can be put into practice. For example, upon starting with the system, an individual (“the person”) can communicate which chronic condition(s) are being managed. The system, which may be automated or may be implemented as a human coach, sets an appropriate outcomes goal for the person, and the person begins using the app. Data monitoring and aggregation collects past recorded data shared by the person, ongoing passively collected data, and/or data logged by the person. Periodically, the Outcomes Model is used to forecast the person's outcomes metric, and the Optimizer is used to select the most effective self-care mode to focus on. A combination of human and/or automated interventions are initiated, focusing on the recommended mode, possibly including use of an Adaptive Support Model. After an appropriate period, the optimization of the outcomes model can be repeated with up-to-date information, and the focus may either be maintained, or switched to a new, now-most-optimal choice. In any case, if the recommended focus is not practical to address for any reason, the next-most-effective mode may be selected. In this way, the system can support people in progressing toward better health outcomes in the way that will be most effective for each person.

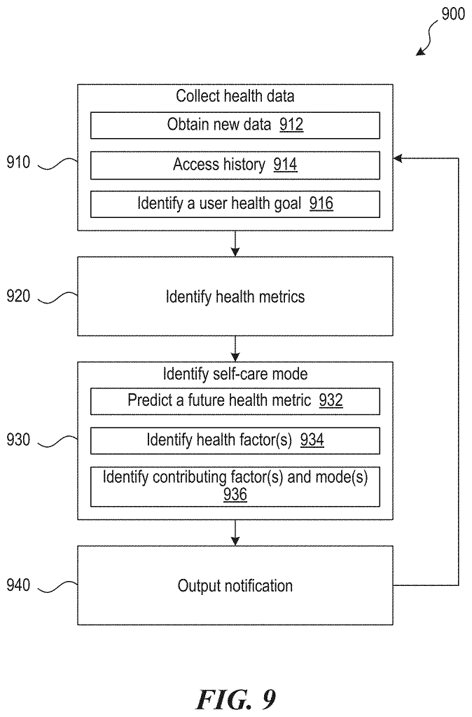

FIG. 9 is a flowchart illustrating a method 900 for selecting a self-care mode, in accordance with embodiments of the present technology. The method 900 can be performed by any of the systems and devices described herein. For example, some or all of the steps of the method 900 can be performed by the system 100 and/or the user devices 104 or analyzing devices 102 of FIG. 1. In some embodiments, the method 900 is performed by a computing system or device including one or more processors and a memory storing instructions that, when executed by the one or more processors, cause the computing system or device to perform one or more of the steps described herein.

The method 900 can be used to determine or predict any of the health metrics described herein. For example, the health metrics can include metrics related to any of the following: blood pressure, blood glucose (e.g., 30-day time-in-range and/or other time-in-range metric), weight, BMI, waist circumference, body fat percentage, cholesterol, triglycerides, heart rate, respiratory rate, body temperature, gases (e.g., oxygen, carbon dioxide), electrolytes (e.g., bicarbonate, potassium, sodium, magnesium, chloride, lactic acid), BUN, creatinine, ketones, alcohols, neurotransmitters, amino acids, hormones, disease biomarkers (e.g., cancer biomarkers, cardiovascular disease biomarkers), and/or combinations thereof. The prediction can be made for any suitable time period, such as at least one week, two weeks, three weeks, four weeks, one month, two months, three months, four months, five months, six months, seven months, eight months, nine months, ten months, 11 months, or 12 months in the future from the date of the prediction.

At block 910, the method 900 begins with collecting health data of a user. For example, at block 912, the system 100 can obtain new data (e.g., real-time and/or most recent set) from the user devices 104. Also, at block 914, the system 100 can access the user history 124 based on interacting with the database 106 of FIG. 1.

As discussed above, the health data (e.g., the new data and/or the user history 124) can include any data relevant to the user's health state. Examples of health data include, but are not limited to, any of the following: blood pressure data (e.g., current and/or previous measurements of systolic and/or diastolic blood pressure), blood glucose data (e.g., current and/or previous blood glucose measurements, current and/or previous HbA1c data values), heart rate data, food data (e.g., number of meals; timing of meals; number of calories; amount of carbohydrates, fats, sugars, etc.), medical history data (e.g., current and/or previous weight, height, BMI, age, sleeping patterns, medical conditions, cholesterol levels, diabetes type, family history, user health history, diagnoses, blood pressure, etc.), activity data (e.g., time and/or duration of activity; activity type such as walking, running, swimming; strenuousness of the activity such as low, moderate, high; etc.), personal data (e.g., name, gender, demographics, social network information, etc.), medication data (e.g., timing and/or dosages of medications such as insulin, prescription and/or non-prescription medications taken), and/or any other suitable data (e.g., basal energy consumption, oxygen consumption) or combination thereof. The health data can be obtained automatically by one or more biosensors (e.g., the biosensor(s) 104a of FIG. 1), input manually by the user (e.g., via the user devices 104), retrieved from a database and/or server (e.g., database 106), or any other suitable collection technique.

Optionally, at block 916, the system 100 can receive a health goal for the user. The health goal can be a target value and/or range for a particular health metric (e.g., blood pressure, blood glucose, weight, etc.) that the user wishes to achieve in the future (e.g., one, two, three, four, five, six, or more months in the future). For example, the health goal can be for the user's health metric to achieve a target value and/or range, to be greater than a target value and/or range, to be less than a target value and/or range, etc. The health goal can be determined by the user, by a healthcare professional, set based on healthcare guidelines (e.g., based on the user's characteristics), or suitable combinations thereof. The health goal can be input by the user via a user device, transmitted to the system from a healthcare professional's device, retrieved from a database or server, or any other suitable technique. Optionally, block 910 can include receiving multiple health goals for multiple different health metrics.

At block 920, the method 900 continues with identifying health metrics from the health data of block 910. The health metrics can be determined from the health data using any suitable approach. For example, as discussed above with reference to FIGS. 2-8, the health metrics can include values (e.g., the most recent value or set of values) and/or statistics (e.g., averages, standard deviations, ranges, sums, differences, ratios, maximums, minimums, percentiles, probabilities, cross-correlations, time-in-range values) for any suitable health data, including, but not limited to, any of the following: blood glucose levels, blood pressure levels, heart rate, weight, food intake, medical history, demographics, diagnoses and/or medical conditions, medications, meditation, sleep patterns, activity patterns, and/or combinations thereof. Health metrics can be computed across health data obtained over any suitable length of time (e.g., 15 days, 30 days, 60 days, or 90 days) and at any suitable time period before the prediction is made (e.g., immediately before the prediction, 15 days before the prediction, 30 days before the prediction, 60 days before the prediction, or 90 days before the prediction).

At block 930, the method 900 can identify a self-care mode for the user. The self-care mode can be a blood pressure reduction mode, cardiovascular risk reduction mode, blood glucose management mode, or another mode. For example, the blood pressure reduction mode can include a set of instructions or a program for reducing systolic blood pressure, limiting blood pressure variability, etc. The cardiovascular risk reduction mode can include one or more dietary programs, exercise programs, tobacco usage monitoring, stress monitoring (e.g., periodic questionnaires, monitoring via a mobile application, etc.), sleep monitoring, and other actions discussed herein. In some embodiments, the cardiovascular risk reduction mode is configured to achieve a maximum reduction of the users cardiovascular risk. The blood glucose management mode can include coordinated operation with a biosensor collecting user health data. For example, the users smartphone can collect glucose level data from the biosensor and provide user actions based on the collected data.

At block 932, the system 100 can generate a prediction (e.g., e.g., forecast) of a health metric of the user. The prediction can be generated using one or more prediction models, e.g., as previously discussed and illustrated with reference to FIGS. 2-8. For example, the prediction can be generated by inputting at least some of the health data or health metrics, into the prediction model(s). In some embodiments, the prediction model(s) are or include one or more machine learning models (e.g., a Gradient Boosted Trees model). In such embodiments, the machine learning model(s) can be trained on health data or metrics from a plurality of different users. The training data may include data for the particular user for which the prediction is to be generated, or may not any include any data from the particular user. As previously discussed, the use of training data from a large number of users allows accurate predictions to be made even for users with limited, irregular, and/or incomplete health data.

The prediction can provide an estimated value and/or range for the health metric at a future time point. The future time point can be at least one or more weeks or months from the date of the prediction. Alternatively or in combination, the prediction can provide an estimated probability that the health metric will achieve a particular target value and/or range at the future time point. In such embodiments, the predicted probability can be expressed quantitatively (e.g., an x % chance of achieving the goal) and/or qualitatively (e.g., highly likely, likely, unlikely, highly unlikely).

At block 932, the system 100 can identify at least one health factor (e.g., blood pressure, blood glucose, heart rate, food, activity, sleep, weight, medication, diagnosis, or demographics, family health history) that contributed to the prediction. The health factor(s) can be identified in various ways, such as by selecting one or more self-care modes that provided a threshold contribution to the prediction, then determining the health factor(s) associated with self-care modes. For example, as previously discussed with reference to FIGS. 2-8, the contribution of at least some of the health factors of a self-care mode can be determined using an optimization algorithm (e.g., a Greedy algorithm) or other suitable technique.

Based on the determined contributions, the system 100 can identify one or more health factors of one or more self-care modes determined to have contributed to the prediction (e.g., self-car modes whose contribution met a threshold value and/or other suitable criteria) as illustrated in block 936. For example, the system 100 can identify one or more (e.g., a predetermined quantity, a number of factors that satisfy a threshold percentage, etc.) self-care modes having the greatest contribution(s) to the prediction, e.g., by ranking the self-care modes in order of contribution magnitude. As another example, self-care modes can be identified based on the percentage and/or proportion of the contribution made to the prediction, e.g., all feature groups contributing at least 10%, 25%, 50%, or 75% to the prediction; feature groups that collectively account for at least 50%, 75%, 90%, or 95% of the prediction; and so on.

At block 940, the method 900 can include outputting a notification to the user. The notification can include the recommended self-care mode, the prediction of the health metric, the at least one health factor determined to have contributed to the prediction, or a combination thereof. For example, if a user's blood pressure is higher than a threshold, method 900 can determine that a self-care mode which includes exercise has a prediction that will lower the user's blood pressure. The notification can include a support message or other feedback informing the user that the predicted outcome can be at least partially attributed to the user's blood pressure levels. The notification can be provided in any suitable format, such as textual, visual, graphical, audible, and/or other formats. The notification can be output to the user via a user interface on a user device or any other suitable computing device.

The method 900 can determine the self-care mode based on identifying a recommended action for the user to improve their health metrics, based on the prediction and/or contributing health factor(s). For example, if the method 900 determines that a certain health factor is particularly influential in causing the user to achieve or not achieve their health goal, the notification can inform the user with recommended actions in a self-care mode with respect to that health factor (e.g., decreasing blood glucose levels; increasing physical activity; improving sleep patterns; altering dietary intake; etc.).

For the data collected after outputting the notification, the system 100 can analyze the collected data to determining user compliance with the output notification. For example, the system 100 can identify user action based on the collected data, such as by estimating exercise events based on the heartrate and accelerometer readings, intake events and nutritional effects (e.g., representative of healthy or unhealthy eating) based on the blood glucose level changes, sleep times or patterns based on heartrate, breathing patterns, accelerometer readings, etc.

The system 100 can use the identified actions to identify a behavior as a set of repeated actions. The system 100 can compare the user behavior to the recommended self-care mode. When the user behavior is closer to the recommended self-care mode than before the recommendation, the system 100 can compare the previous health data to health data collected after the recommendation. Additionally or in combination, the system 100 can derive a trend in the health metrics. When the change or the trend over a predetermined duration does not satisfy a threshold, the system 100 can identify and output a new self-care mode based on the process described above. When the user behavior does not change beyond a threshold requirement, the system 100 can determine that the user is unresponsive to the output notification. Such determination can trigger the system 100 to identify a different self-care mode and/or adjust an output detail (e.g., an urgency in the communication, a frequency in a rewarding/encouraging mechanism, a change in output format, or the like). Subsequent user reaction to the changes can further be logged and analyzed as described above.

Accordingly, the recommendation can be customized to the particular user, e.g., based on user feedback, behavioral patterns, etc. For example, the method 900 can account for whether the user has historically complied or not complied with a particular lifestyle change, whether the user has expressed a preference for certain types of behavioral interventions, etc. Such information can also be used as input for generating future recommendations and/or other notifications to assist the user in meeting their health goals.

In some embodiments, the approaches described herein provide improved accuracy in predicting various health metrics (e.g., 30-day time-in-range, 30-day average blood glucose values, 30-day average blood pressure values, weight, etc.) compared to conventional techniques. For example, the RMSE of predictions generated in accordance with embodiments of the present technology can be no more than 20%, 15%, 10%, or 5%. The relative reduction in RMSE compared to a naïve predictor (e.g., a persistence model) can be at least 10%, 15%, 20%, 25%, 30%, 40%, or 50%. The enhanced accuracy can assist users in monitoring and managing their health conditions, as well as in making decisions regarding lifestyle changes and/or other actions to improve their health.

The system 100 can provide the improved accuracy in predicting the metrics and improve the user's behavior in managing their health conditions by transforming the collected data into contextual goals and metrics. The context-based results can be used to predict the future outcomes, and the further outcomes can be further analyzed to identify the contributing factors. Thus, the system 100 can transform the measured values into specific actions and output the corresponding message to the user. The user response can be further identified from subsequently collected data, and the user response can be used to further transform the self-care mode recommendation and/or the models used to implement the processes described above.

In some embodiments, collected data, behaviors, reaction or response patterns, or the like can be grouped across multiple users having one or more shared characteristics. Accordingly, the system 100 can crowdsource the data and leverage improvements of other users having similar health conditions, personalities, contexts, or other contributing factors.

The system 100 can generate an adaptive score for the risk of developing one or more diseases, such as cardiovascular disease (CVD), within a time period (e.g., 1 year, 10 years, etc.). The score can be based on a risk score (e.g., InterHeart risk score), but any other well-established score may be used with the appropriate modifications. The system 100 can adjust the score by adding inputs not currently included in the risk score but which are known to be risk factors for the disease or by replacing discrete inputs to the risk score with new factors that are continuous, so that the new risk score changes over time as new inputs from sensors are obtained. The system 100 can determine a risk score based, at least in part, on blood glucose data as discussed in connection with FIGS. 5, 10, and 11, blood glucose levels as discussed in connection with FIGS. 4 and 5, blood pressure data (e.g., systolic blood pressure variability) discussed in connection with FIGS. 6, 7, and 8, and/or other input, scores, or data discussed herein. The system 1000 can continuously or periodically recommend self-care modes based on new data, user settings, or other input to adaptively provide recommendations. The system 1000 can recommend a self-care mode in an SMS message, email, a notification in an application on a user interface, or any notification method on a user device, and can integrated with other applications (e.g., mobile applications) or user devices.

For cardiovascular disease, the scoring system (e.g., InterHeart scoring system) can apply an additional percentage increase (e.g., 12% increase) in CVD risk for each point, and provides for cardiovascular (“CV”) risk scores ranging from 0 to 40 points. In such a scoring system, the risks are relative to the risk of a person without any risk factors. For example, such a person will have a CV risk score of 0 (zero “points”), and a relative risk of 1 (=1.12⁰). For someone with 20 points, the risk is about 9.64 (1.1220) times higher than someone without any risk factors. Someone who scores the maximum number of points (40) can have just over 93 times the risk of someone with no risk factors.

The system 100 can make adjustments by using body mass index (BMI) (e.g., instead or in conjunction with waist-hip ratio) as a risk factor, defined as the weight in kilograms divided by the square of individual's height in meters. By converting the risk associated with BMI to align with the used scoring system, the system 100 can apply in line with literature no risk points when BMI is below 26 kg/m² and (BMI-26)/1.7 points for BMI values above that level, capped at a number of points (e.g., 8 points). With no height changes expected in adults, the system 100 can compute the BMI with every new weight entry and adjust the heart risk score accordingly. The system 100 can make adjustments by using blood-pressure medication information to reduce the risk score for people taking such medications routinely. The change in score differs for those with diabetes and those without, such that taking blood-pressure medications reduce the score for people with no diabetes by 2.083 points, and for those with diabetes condition only by 1 point.

In some embodiments, the system 100 can make adjustment by using a continuous score, based on increased risk. For people with no known diabetes, the system 100 can convert the associated increased risk, given as a hazard ratio of ranges of a1c values, into the following risk scores that depend on the variable x representing user's 30-days average BG values:

$$s = \{ \begin{matrix}0.00746 {x}^{2} - 1.119 x + 41.96 , & for 65 \leq x < 75 \\ 0 , & for 75 \leq x < 110 \\ 0.04428 x - 4.871 , & for 110 \leq x < 141 \\ \frac{12}{1 + {exp}^{- \frac{\frac{x}{0.209} - 997.7}{159.89}}} , & for x \geq 141\end{matrix} \qquad Equation 1$$

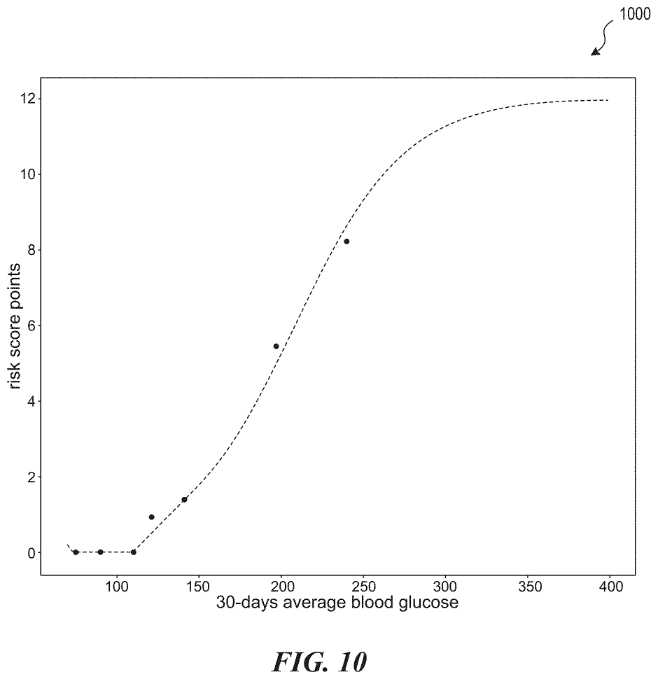

Example 1000 of FIG. 10 illustrates the blood glucose adjustment for people without known diabetes. The function shown in Equation 1 was computed by solving a constrained optimization problem, where the a1c values were first converted to 30-days blood glucose (BG) average via b̅g̅=28.7×α1c−46.7. The points to fit were the middle of each a1c range for which the risk was reported, with values set as the hazard risk converted into risk points via

$$\frac{log ( r )}{log ( 1.12 )} ,$$

where r is the relative risk. For the range over the normal range of 110 mg/dL, the constraints included setting the upper bound of the function at 12 points, and fitting the function:

$$s = \{ \begin{matrix}{v}_{1} x - {v}_{2} , & for 110 \leq x < 141 \\ \frac{12}{1 + {exp}^{- \frac{\frac{x}{{w}_{1}} - {w}_{2}}{{w}_{3}}}} , & for x \geq 141\end{matrix} \qquad Equation 2$$

The value of the derivative of the part where x≥141 equals vi at the point, to x=141, allow for a smooth function. The part with average BG values lower than the normal (75 to 110 mg/dL) was selected by fitting a quadratic function, as a single risk value and an anchor at x=75 were available. For example, the system 100 used the iterative dogleg algorithm with rectangular trust regions.

For people with known diabetes the relative risk is higher everywhere on the BG spectrum, and converges to the same risk around 300 mg/dL average BG. System 100 can user four points of reference reported for the cardiovascular risk of people with diabetes4, where three points refer to the center of bins under study, and the fourth constrains the function to converge to the same value as for people without known diabetes at very high BG levels. The system 100 uses the same family of functions to fit those points by minimizing the squared error for.

$$s = \frac{12}{1 + {e}^{- \frac{\frac{x}{{w}_{1}} - {w}_{2}}{{w}_{3}}}} \qquad Equation 3$$

The overall new score function is:

$$\begin{matrix}s = \{ \begin{matrix}0.007 {x}^{2} - 1.1 x + 41.9 , & no diabetes 65 < x \leq 75 \\ 0 , & no diabetes 75 < x \leq 110 \\ 0.045 x - 4.87 , & no diabetes 110 < x \leq 141 \\ \frac{12}{1 + \text{?}} , & no diabetes x > 141 \\ \frac{12}{1 + \text{?}} , & diabetes\end{matrix} & Equation 4 \\ \text{?} \text{indicates text missing or illegible when filed} & \end{matrix}$$

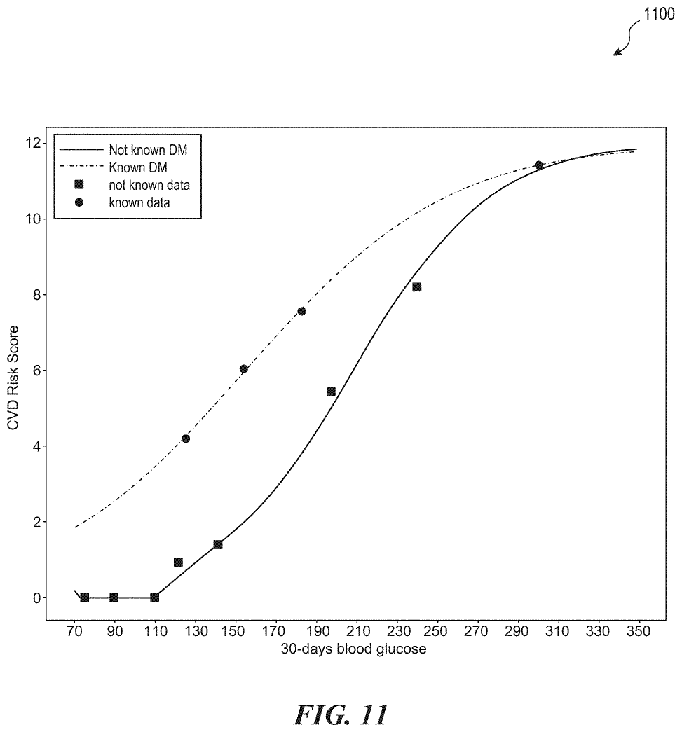

The resulting adjustment functions in terms of risk scores for people with and without known diabetes are shown in example 1100 of FIG. 11. FIG. 11 illustrates a blood glucose adjustment for people with (dotted line) and without (solid line) known diabetes. The constraining points based on the corresponding studies are shown in the plot.

The system 100 can assigns a set value (e.g., 3, 4, 5 values/points) for having high-blood pressure. A continuous score is generated based on increased risk observed in research studies and meta analyses, encompassing together observations from more than a set number of participants (e.g., 500,000, 750,000, 1,000,000 participants). The associated increased risk is converted, given as a risk ratio between levels of systolic blood pressure, into the following risk scores that depend on the variable x representing user's 30-days average BP value:

s=0.0000699·x³−0.0303·x²+4.46809·x−220.38

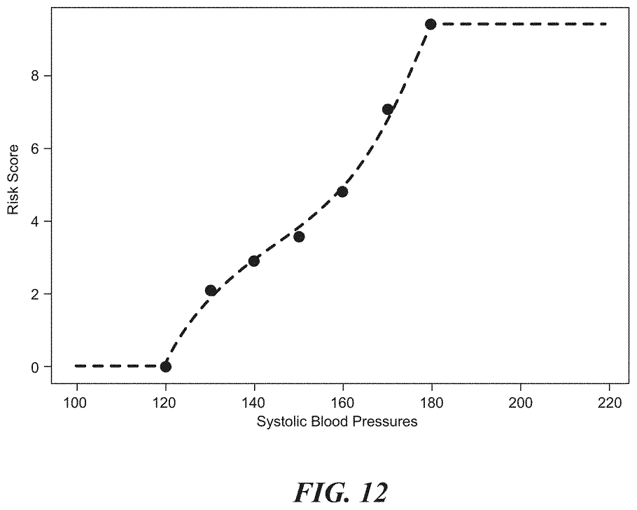

For systolic blood pressures values between 120 and 180. Below threshold a blood pressure (e.g., 110, 120, 130) there is no increased CVD risk, yielding a score of zero, and above 180. The system 100 can set a score to be equal to that at 180 mmHg. A plot of the function is shown in FIG. 12. Blood pressure variability can be a risk factor for CVD independent from blood-pressure levels. The system 100 can add risk scores based on blood pressure variability, exercise data, sleep data, dietary data, etc. The added risk score can be increased or decreased based on non-blood pressure data, such as diet.

| | Added |
|---|---|
| BP variability | risk score |
| <6.5 mmHg | 0 |
| Between 6.5 | 1.68 |
| and 8.7 mmHg | |
| Between 8.7 | 2.04 |
| and 11.0 mmHg | |
| Between 11.0 | 2.11 |
| and 14.4 mmHg | |

The system 100 can generate an adaptive score for the risk of developing a cardiovascular disease (CVD) based on blood pressure-related data. The system 100 can use health data selected for the user to generate the CV risk scores or other risk scores. Composite and aggregate scoring can be used with scoring functions and use to identify one or more self-care modes or actions. Below are example user answers and assigned risk scores, which can be used as inputs.

| | |
|---|---|
| Answer | Score |

Age/Sex

| | |
|---|---|
| Male, older than 55 | 2 |
| Female older than 65 | 2 |
| Other | 0 |

Diabetes

| | |
|---|---|
| Answer | Score |
| Yes | 6 |
| No | 0 |

High Blood Pressure

| Yes | 5 |
|---|---|
| No | 0 |
| Family History | |
| Q: Have either or both of your | |
| biological parents had a heart attack? | |
| Yes | 5 |
| No or Unsure | 4 |
| Tobacco Usage | |
| Q: Which best describes your | |
| history of tobacco use? | |
| Smoking 1-5 cigs/day | 2 |
| Smoking 6-10 cigs/day | 4 |
| Smoking 11-15 cigs/day | 6 |
| Smoking 16-20 cigs/day | 7 |
| Smoking 21+ cigs/day | 11 |
| Former Smoker | 2 |
| None | 0 |
| Q: Over the past 12 months | |
| what has been your typical | |
| exposure to other people's smoke? | |
| Yes, at least 1 hour/week | 2 |
| No or less than 1 hour/week | 0 |
| Stress | |
| Q: How often have you felt | |
| stress in the past year? | |
| Never Experienced Stress | 0 |
| Several Periods of Stress | 0 |
| Some Period of Stress | 3 |
| Permanent Stress | 3 |
| Depression | |
| Q: During the past twelve months, was | |
| there ever a time when you felt sad, blue, or | |
| depressed for two weeks or more in a row? | |
| Yes | 3 |
| No | 0 |
| Physical Activity | |
| Q: How active are you during | |
| your leisure time? | |
| Mainly sedentary (e.g., sitting, | 2 |
| reading, watching television) | |
| Mild exercise, minimal effort | 2 |
| (e.g., yoga, archery, sport fishing, | 0 |
| easy walking) | |
| Moderate exercise (e.g., walking, | 0 |
| bicycle riding, or light gardening | |
| at least 4 hours per week) | |
| Strenuous exercise (heart beats | 0 |
| rapidly e.g., running/jogging, | |
| football, vigorous swimming) | |
| Diet | |
| Q: Do you eat salty food or snacks one or | |
| more times a day? | |
| Yes | 1 |
| No | 0 |
| Q: Do you eat deep fried foods or snacks or | |
| fast foods 3 or more times a week? | |
| Yes | 1 |
| No | 0 |

Q: Do you eat fruit one or more times daily?

| Yes | 0 |
|---|---|
| No | 1 |
| Q: Do you eat vegetables one | |
| or more times daily? | |
| Yes | 0 |
| No | 1 |
| Q: Do you eat meat and/or poultry two | |
| or more times daily? | |
| Yes | 2 |
| No | 0 |
| Physical Measurements | |
| Q: Waist/Hip Ratio | |
| Below 0.87 | 0 |
| Between 0.87 and 0.97 | 2 |
| Above 0.97 | 4 |

The system 100 can use subsets for data to generate inputs, such as risk scores, such as cardiovascular risk score, cancer risk score, etc. The risks scores can be adjusted based on family history, genetic information, or other information provided by, for example, the user, healthcare provider, etc.

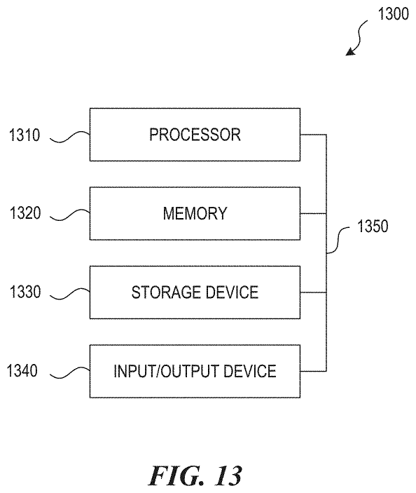

FIG. 13 is a schematic block diagram of a computing system or device (“system 1300”) configured in accordance with embodiments of the present technology. The system 1300 can be incorporated into or used with any of the systems and devices described herein, such as the system 100 and/or user devices 104 or analyzing devices 102 of FIG. 1. The system 1300 can be used to perform any of the processes or methods described herein with respect to FIGS. 1 and 2. The system 1300 can include a processor 1310, a memory 1320, a storage device 1330, and an input/output device 1240. Each of the components 1310, 1320, 1330 and 1340 can be interconnected using a system bus 1350. The processor 1310 can be configured to process instructions for execution within the system 1200. In some embodiments, the processor 1310 can be a single-threaded processor. In alternate embodiments, the processor 1310 can be a multi-threaded processor. Although FIG. 12 illustrates a single processor 1310, in other embodiments the system 1300 can include multiple processors 1310. In such embodiments, some or all of the processors 1310 can be situated at different locations. For example, a first processor can be located in a sensor device, a second processor can be located in a user device (e.g., a mobile device), and/or a third processor can be part of a cloud computing system or device.

The processor 1310 can be further configured to process instructions stored in the memory 1320 or on the storage device 1330, including receiving or sending information through the input/output device 1340. The memory 1320 can store information within the system 1300. In some embodiments, the memory 1320 can be a computer-readable medium. In alternate embodiments, the memory 1320 can be a volatile memory unit. In yet some embodiments, the memory 1320 can be a non-volatile memory unit. The storage device 1330 can be capable of providing mass storage for the system 1300. In some embodiments, the storage device 1330 can be a computer-readable medium. In alternate embodiments, the storage device 1330 can be a floppy disk device, a hard disk device, an optical disk device, a tape device, non-volatile solid-state memory, or any other type of storage device. The input/output device 1340 can be configured to provide input/output operations for the system 1300. In some embodiments, the input/output device 1340 can include a keyboard and/or pointing device. In alternate embodiments, the input/output device 1240 can include a display unit for displaying graphical user interfaces.

Non-transitory computer program products (i.e., physically embodied computer program products) are also described that store instructions that, when executed by one or more data processors of one or more computing systems, cause at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

The systems and methods disclosed herein can be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Alternatively or in combination, the display device can be a touchscreen or other user input device configured to accept tactile input (e.g., via a virtual keyboard and mouse). Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The technology described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

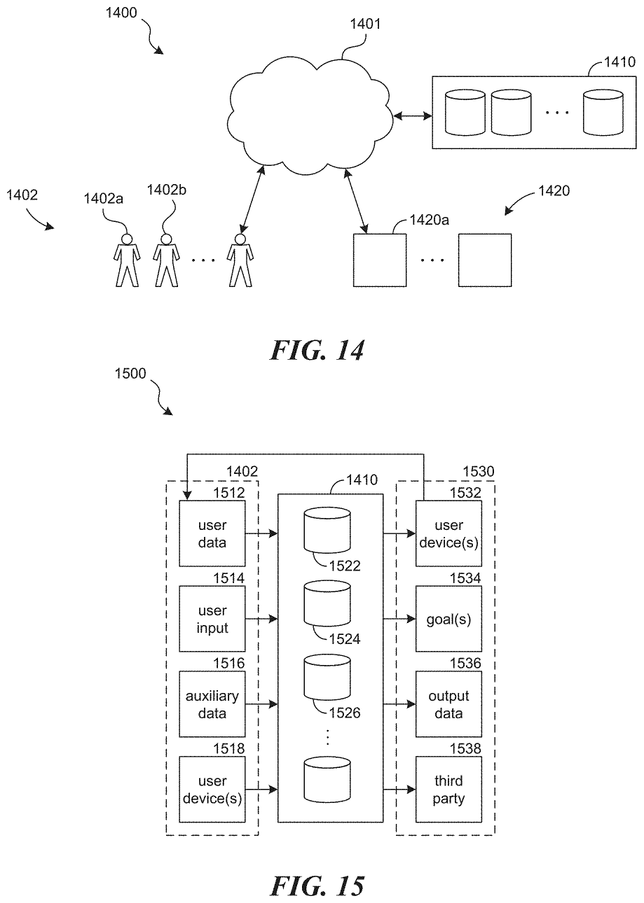

The computing system can include clients and servers. A client and server are generally, but not exclusively, remote from each other and typically interact through a communication network. The relationship of client and server arises by virtue of computer programs running on the respective computers and having a client-server relationship to each other. The computing system can be part of or incorporated into the systems discussed in connection with FIGS. 14 and 15.

FIGS. 14 and 15 are schematic diagrams illustrating exemplary computing environments in which a healthcare guidance system operates, in accordance with embodiments of the present technology. A system 1400 can include a network 1401, a biomonitoring and healthcare guidance system 1410 (“system 1410”), users or user devices 1402 (“user devices 1402”), and additional systems 1420. The network 1301 can transmit data between the user devices 1402, healthcare guidance system 1410, and/or additional systems 1420. The system 1410 can include analyzing devices select one or more databases, models, and/or engines to analyze received data to provide self-care modes, predictions, identify contributing health factors, or the like. The description of system 100 of FIG. 1 applies equally to the system 1400 unless indicated otherwise, and the system 1400 can perform the methods disclosed herein.

The system 1410 can include databases, models, systems, and other features disclosed herein and can include models, algorithms, engines, features, and systems disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; PCT App. No. PCT/2021/028445, and other patents and applications discussed herein. For example, the system 1410 can receive health data (e.g., glucose levels, blood pressure, etc.) from user devices disclosed in U.S. application Ser. No. 16/888,105 or U.S. application Ser. No. 17/236,753 and can forecast or predict one or more health metrics disclosed in U.S. application Ser. No. 16/888,105 or U.S. application Ser. No. 17/167,795. The self-care modes can be periodically or continuously generated based on predicted or detected events, user settings, schedules, or the like. Forecasted metrics can be used to determine a behavioral intervention plan, turn-by-turn health plan, etc. In some implementations, the system can receive information about a predicted event, such as a hypoglycemic or hyperglycemic event. The system can identify the event and then periodically (e.g., hourly, daily, weekly, monthly) or continuously (e.g., in response to continuously received CGM data) recommend self-care modes to reduce or avoid the risk of predicted event.

The system 1400 can provide healthcare support in the form of behavioral interventions to achieve exercise goals. For example, the user 1402b can be training to increase cardiovascular health. The system 1400 can receive user exercise data (e.g., workout type, workout duration, etc.), exercise data (e.g., heart rate, blood pressure, etc.), positioning data (e.g., GPS data), or other data. The healthcare guidance system 1410 can use the data (all the data or subset of the data) determine healthcare support actions and behavioral interactions to be performed to, for example, develop behavioral intervention plan for completing workouts. The healthcare guidance system 1410 can use forecasting models or engines to determine recommendations for the user and can generate new models based on newly available data. The forecasting models or engines can be used for multiple users or a single user. In some embodiments, data associated from a user can be inputted into different models or engines and the output from those engines or models can be grouped, processed, and/or feed into additional models or engines, including those disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; and PCT App. No. PCT/2021/028445. For example, the healthcare guidance system 1410 can perform one or more steps of the method 200 of FIG. 2 or method 900 of FIG. 9 using output or data from patents and applications referenced herein.

The network 1401 can communicate with an auxiliary computing system 1420 that can provide programs, or other information used to manage the collection of data. For example, a computing system 1420a can communicate with a wearable user device 1420a to provide firmware updates. The healthcare guidance system 1410 can automatically update databases, models, and/or engines based on changes to the user device 1402a based on the update. The computing system 1422a and healthcare guidance system 1410 can communicate with one another to further refine data analysis.

A user can manage privacy and data settings to control data flow. In some embodiments, one of the computing systems 1420 is managed by the user's healthcare provider so that received user data is automatically sent to the user's physician. This allows the physician to monitor the health and progress of the user. The physician can be notified of changes (e.g., health-related events) to provide further reinforcement monitoring, new health goals, history data, modified health metrics, etc. The healthcare guidance system 1410 can adjust behavioral interventions based on input from the healthcare provider. For example, the healthcare provider can add health care support parameters, such as target goals for losing weight, reducing blood pressure, increasing exercise durations, etc., as well as constraints for optimization routines. The behavioral intervention programs can be modified by the user, healthcare provider, family member, authorized individual, etc.

The healthcare guidance system 1410 can forecast events, predict health states, and/or perform any of techniques or methods disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; and PCT App. No. PCT/2021/028445. For example, the system 1400 can accurately determination of glucose concentration in the blood7 of an individual at a present time and/or in the future and can adaptively provide healthcare support to achieve health goals. The system 1400 can then develop personalized biomonitoring and/or providing personalized healthcare recommendations or information for the treatment of diabetes and other chronic conditions, exercise programs, or the like. Behavioral interventions programs can be used to further assist with user responsiveness.

In some embodiments, the system 1400 can provide each user 1402 with one or more self-care modes. The healthcare guidance system 1410 can collect health user data of the user 1402a and can identify a health metric based on the health data. The system 1410 then identifies a self-care mode from a plurality of self-care modes by, for example, analyzing a plurality of available self-care modes, each having one or more contributing factors, determining one or more predictions of the health care metric based on the self-care modes, identifying contribution factors of the self-care modes for the predictions, and selecting a self-care mode from the plurality of available self-care modes based on the contribution factors. The system 1400 can now put a notification for the user for viewing on a device of the user 1402a. The notification can include prompts, recommendations, self-care actions/programs, predictions, identification of contributing health factors, prompts, or other suitable data. Self-care modes can be recommended continuously or periodically at regular or irregular intervals or in response to an event (e.g., unwanted predicted event, user inputted event, etc.)

If the user 1402a is concerned about cardiovascular health, the system 1400 can determine a cardiovascular risk score and can recommend a self-care mode to reduce or minimize the cardiovascular risk score. The collected health data can include systolic blood pressure data, blood pressure variability, exercise data (e.g., exercise data collected via a wearable device, smart phone, user input, etc.), cholesterol levels, or combinations thereof. When new user device is available, the system 1400 can pair with the newly available device and automatically collect new data that may provide indication suitable for determining the cardiovascular risk score. The system 1410 can receive and analyze the data to determine one or more predictions, such as predicting a minimization reduction of the cardiovascular score. The system 1410 can also identify contributing factors, including diet, exercise, weight of the user, or the like. In some embodiments, the system provides ranking of the contributing health factors to allow the user 1402a to prioritize recommendation adherence, as discussed on connection with FIG. 15.

The system 1400 concurrently provide other support to the user 1402b. If the user 1302b suffers from diabetes, the system 1400 can support a glucose management self-care mode. Blood glucose levels, exercise data, sleep data, and other health data can be transmitted to the system 1410. The system 1410 can analyze the data to predict hypoglycemic events, hyperglycemic events, maximum/minimum blood glucose levels, blood glucose level ranges, or the like. The system 1410 can also identify contributing health factors for the blood glucose levels, including diet, weight, exercise, sleep, or the like. The system 1400 can generate recommendations and corresponding warnings, incentives, prompts, or other notifications. The system can also evaluate whether the user history indicates a higher likelihood of user compliance or suggestions, notifications, and other demonstrations to generate corresponding recommendations.

The system 1400 can monitor the contributing health factors over a period of time to adaptively update the self-care mode and recommendations. When the user 1402b completes an exercise routine, the system can notify the user of changes of contributing health factors. For example, if the user reaches a healthy weight, the system 1410 can notify the users that his/her weight is not a contributing factor for glucose management, thereby informing the user to focus on other contributing factors. When the user completes actions, the system 1400 can assign corresponding positive scores or weights to preceding actions. This allows behavioral intervention to be incorporated into the self-care mode and corresponding recommendations.

In another implementation, users 1402 may be recommended a weight management self-care mode. The system 1400 can collect weight data, body mass index, age, sex (e.g. male or female), or other health data. The guidance system 1410 can predict an increase of the user's weight based on contributing health factors. The system 1400 can recommend dietary changes, sleeping schedule, exercise schedule, and other contributing health factors. The system can also analyze other recommended self-care modes to provide consistent actions. For example, the system 1400 can prioritize predictions for a particular user. This allows contributing health factors to prioritize predictions to be recommended. For example, a user can be recommended both a weight management self-care mode and a glucose management self-care mode. Predictions for weight management self-care mode can be long term weight loss, whereas the predictions for the glucose management self-care can be short-term blood glucose levels. The healthcare guidance system 1410 can provide actions for the self-care mode to keep the user within acceptable blood glucose levels while also providing actions for the weight management self-care mode for weight loss.

The systems disclosed herein can use weighting algorithms, optimization functions, and other routines/functions for providing self-care modes for a group of users (e.g., family members), managing multiple conditions, or other like. In some embodiments, the systems can provide a self-care mode for multiple users. For example, related users may suffer from the same or similar conditions. The self-care mode can be designed to manage the condition(s), such as hypertension, obesity, etc. The allows users (e.g., family members, friends, partners, etc.) to implement a single self-care plan (including dietary program, dietary program, etc.) to improve overall health. The system can notify the users if a single self-care mode is no longer suitable to both users. The system can then send individualized self-care modes to each user.

A self-care mode can be designed for multiple conditions by, for example, using weighting, constrained optimization, averaging function, or the like. In non-dominate condition implementations, multiple conditions can be weighted, scored, and optimized to manage, for example, (1) diabetes and hypertension; (2) diabetes and cardiovascular disease; (3) diabetes and obesity; (4) diabetes, cardiovascular risk, and obesity; etc. A self-care mode for reducing cardiovascular risk can also manage hypertension and blood glucose levels. Predicted blood pressure and blood glucose levels can be weighted and prioritized based on, for example, prioritized user goals, healthcare provider input, healthcare impact score, etc. In some dominate condition implementations, one or more conditions can be identified for constraints used during optimization to reduce/increase one or more objectives as long as another health objective for the dominate condition is above or below a certain threshold. This allows important health conditions to be prioritized.

FIG. 15 is a schematic diagram illustrating an embodiment of the system for providing adaptive healthcare support for a user 1402, in accordance with an embodiment of the present technology. The description of the system 1400 of FIG. 14 applies equally to the system 1500 unless indicated otherwise, and system 1400 can perform the methods disclosed herein.

The system 1500 can collect user data, user input, auxiliary data, etc. The user data can be collected by sensors (e.g., glucose sensors, wearable sensors, etc.), received from a remote computing device (e.g., a cloud platform storing user history data, real-time data, etc.), or other data. The user input can be health data (e.g., weight, BMI, etc.), exercise or motion data (e.g., distance walked, distance run, etc.), goals, achievements, ratings/rankings (e.g., ranked goals, rated activities, etc.), or other data inputted by the user using one or more computing devices, such as a mobile phone, computer, etc. This allows a user to input data that is not automatically collected. The auxiliary data 1516 can be selected by the system 1410 to modify the adaptive support machine-learning model based on received indication of the response. The auxiliary data 1416 can include predictions (e.g., short-term predictions, long-term predictions, forecasted events, etc.), environment data (e.g., weather data, temperature data, etc.), or the like. The auxiliary data 1516 can be inputted to models to generate output data based on non-user specific parameters.

The system 1410 can request auxiliary data or communicate with device(s) to receive data indicative of a past user state, a past action presented to the user, a past user behavior, health status, or combinations thereof. In some embodiments, the system 1410 can establish communication with connected device (e.g., vehicle) associated with the user, IoT hubs (e.g., IoT devices with Google Assistance, Siri, Alexa, etc.), IoT devices (e.g., motion sensors, cameras, etc.), surveillance systems, etc. For example, when a user arrives home after work, the user may not be receptive to certain prompts for a period of time. The system 1410 can receive auxiliary data (e.g., a garage door opening, surveillance system turned OFF, etc.) indicating when the user returned home. The system 1410 can determine a program or a set of delivery details for adjusting a content and/or a delivery timing for recommended actions based on the user's arrival time. The system 1410 can adaptive request and receive data from different sources to adaptively train the models and engines disclosed herein. The system 1410 can manage identification and authentication for integration with auxiliary platforms, devices, and systems. In some applications, the system 1410 can incorporate weather data to maximize behavior intervention by, for example, providing prompt (e.g., prompts to exercise outside, walk, etc.) suitable for the weather conditions. Health predictions can be considered to develop behavioral interventions designed to increase health scores for the user, enhance goal setting, and accurately identify self-care modes or user states.

The user input 1514 can include one or more new goals, such as maintaining glucose levels, losing weight within a set period or time, answers to questions, risk scores, etc. The guidance system 1410 can select databases (e.g., pooled user data) and models for recommending user device(s) for collecting target data, analyzing the one or more new goals, recommending user device(s) for reinforcements, etc. The guidance system 1410 can send the information (e.g., future health metrics, self-care modes, alternative self-care modes, etc.) to user device 1532 for viewing by a healthcare provider or third-party device 1538, or third-party device 1420 as discussed in connection with FIG. 14.

The system 1410 can receive one or more user history items associated with the user 1402. The user history items can define a past user state, a past action presented to the user, a past user behavior, or combinations thereof. The system 1410 can select an adaptive healthcare support engine 1522 trained to estimate user information, such a current state or predicted state of the user, based on the one or more user history items. The system 1320 can utilize the adaptive healthcare support engine 1522 or another engine 1524 to identify one or more actions for the user based on the user information. The user device(s) 1518, 1532 can execute the one or more identified actions for the user and can receive an indication of a behavior of the user performed in response to the action. The system 1410 can update one or more of the adaptive support models (e.g., models 1522, 1524, 1526, etc.) based on the received indication of the behavior detected by the user devices 1518 or 1432, or indicated by the user.

In some embodiments, the system 1410 can receive new data from the user 1302. The new data can represent health sensor data, a biometric condition, user input data, a user motion, a user location, or a combination thereof. The health sensor data from a user device 1518 can include glucose levels, blood pressure, heart rate, analyte levels, or other detectable indicators of the state of the user. The system 1410 can access one or more user history items (e.g., items stored in database 1426) defining at least one of a past user state, a past action presented to the user, and a past user behavior. The past user state can represent a physiological or a health condition of the user occurring or processed at a past time. The past action can represent a previously identified action taken by the user. The past user behavior can represent a repeated action occurring with a temporal pattern. The actions can be detected or identified by user device(s) 1518, 1532, or another suitable means, such as biomonitoring devices or via user input 1514.

The system 1410 can estimate a recent state of the user based on the new data and the one or more user history items. The recent state represents a current or a recent health condition of the user (e.g., most recent health condition, health condition within a predetermined period of time, etc.). The health condition can be, for example, hypoglycemic state, hyperglycemic state, high blood pressure, etc. The system 1410 can determine a likely outcome (e.g., increase/decrease in glucose levels, blood pressure, etc.) based on the recent state for represent a thresholding health condition of the user likely to occur at a future time. The system 1410 can then identify one or more actions for the user based on the recent state using one or more adaptive support machine-learning models. The actions can be sent to the user devices 1532 for user notification to affect a targeted user action before the future time to prevent or adjust the likely outcome. In some embodiments, the identified actions are selected based on whether the user devices 1532 is capable of identifying the action. For example, if the user has wearable exercise monitor, the identified actions can include exercises detectable by the wearable exercise monitor. In some embodiments, the user can be prompted to input whether the action has been completed. The system 1410 can also provide goal(s) 1534, output data 1536, or other information disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; and PCT App. No. PCT/2021/028445.

The system 1410 can also determine a set of delivery details for adjusting a content and/or a delivery timing for the recommended action. The user device(s) 1532 can execute the identified action according to the set of delivery details. The system 1500 can receive or identify and indication of a response of the user performed in response to the action. When the response corresponds to the past user behavior, the system 1410 can update associated adaptive support machine-learning models based on the received indication of the response. The system 1410 can add engines and models based on newly available data, new users, or the like to provide adaptability.

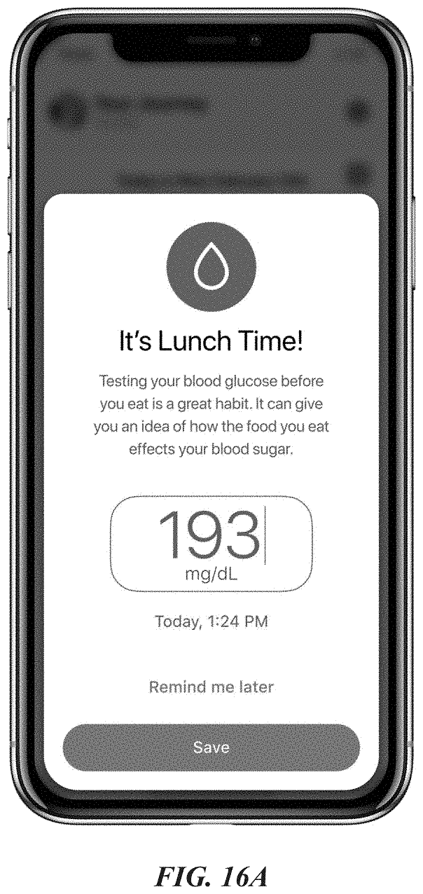

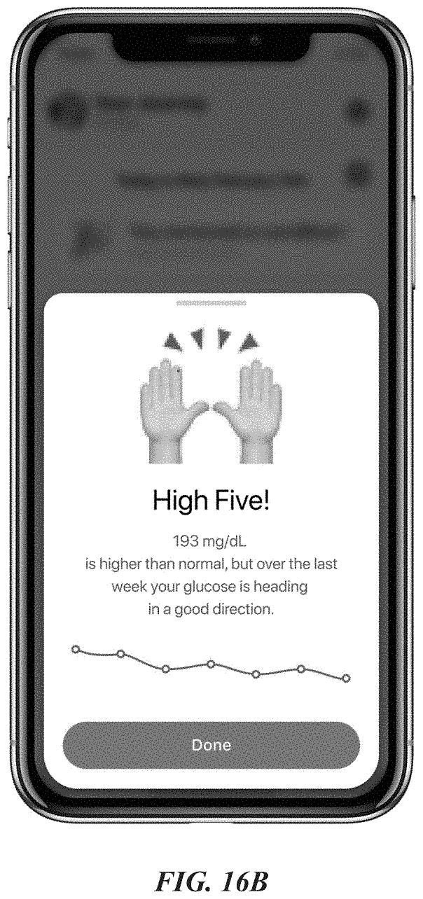

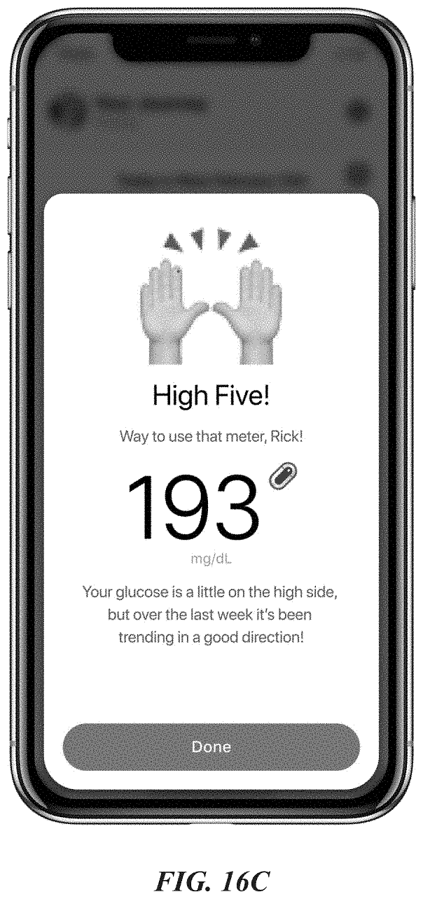

FIG. 16A-16C illustrate examples of prompts (FIG. 16A) and reinforcements (FIGS. 16B, 16C) output by a biomonitoring and healthcare guidance system configured in accordance with embodiments of the present technology. The systems disclosed herein can send notifications for supporting a single target behavior as part of the self-care mode by, for example, sending prompts, messages, reinforcements, and notifications. The systems disclosed herein can continuously or periodically identify self-care goals to, for example, optimize overall health, manage a health condition, avoid or reduce the risk of the predicted event, mitigate the risk of developing a disease or condition, combinations thereof, or the like. A user can select the frequency of self-care mode identification, prompts, notifications, and other interaction with the systems. In some embodiments, the user can schedule notifications of self-care modes to increase behavioral change. For example, the user can schedule an exercise self-care mode before beginning an exercise routine. When the user receives notification that exercise is a significant contribution factor to overall health, the user may be motivated to exercise more strenuously or for a longer period of time.

The systems may further utilize warnings or messages having varying degrees of urgency or impact. A target behavior of a self-care mode can be, for example, a single action (a task) which may be performed regularly, such as taking medication, checking blood sugar or blood pressure, or similar discrete, repeated actions. The system can receive data on an individual's past performance of the task, past prompt and reinforcing messages sent to the individual, and/or related information about the individual. The system can input that information to a trained model, which determines when to send prompt or reinforcement messages so as, over time, to bring the individual's rate of task completion toward a target rate.

In embodiments where the self-care mode provides a task in the form of a discrete action, each day can be divided into a number of periods. At the start of each period, the model can calculate whether or not to send a prompt message. During the period, the individual either performs the task, or does not. The model can calculate whether or not to send a reinforcement message if the individual performs the task during the period.

From day to day and over time, a single individual may change in their responsiveness to prompts and reinforcements. A prompt rate that was helpful at one point can become easy to ignore later on, or may become annoying. Different individuals may also react differently to prompts and reinforcements. Moreover, different individuals may react differently according to the urgency of the content, the type of communication (e.g., warnings or prompts), output format (e.g., audible or visual, emphasizing numbers or graphics, etc.), the timing of the message relative to a desired timing of the response, or the like. Accordingly, one goal of the optimization at each period can be to determine whether, for that individual at that time, a prompt, reinforcement, or both would be effective or not at getting the individual to do the task at the desired rate. Also, the optimization can determine the urgency, the type, the timing, etc. associated with delivering the message or recommendation.

In some embodiments, at each period, the goal of the calculations may not simply be to maximize the probability of task completion in the following period, but rather, to increase or maximize the expected discounted rate of task completion into the future. In such embodiments, messaging that results in high rates of task completion for a short time but then no further task completion may be considered less successful than messaging that results in building the individual's task completion rate up to the desired level and maintaining it there for a long period of time. In short, the model's decisions can be aimed at optimizing the overall task completion by the individual over time, with more emphasis on task adherence in the near future.

In some embodiments, an adaptive support model can be configured to learn from a multitude of users while keeping track of notifications, messages, and/or behavior of each user over time, in order to serve them with the optimal messages at every time slot. The model may use a definition of (a) a vector describing the state (e.g., the health state or the current activity) of an individual at a given time, (b) a list of possible actions the system can take, and/or (c) a reward function. The systems disclosed herein can include the technology and features disclosed in U.S. application entitled SYSTEMS FOR ADAPTIVE HEALTHCARE SUPPORT, BEHAVIORAL INTERVENTION, AND ASSOCIATED METHODS, filed Jun. 3, 2021 (Attorney Docket No. 137553.8018.US01), listing Ydo Wexler al. as inventors can be part of the systems disclosed herein.

### CONCLUSION

The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the embodiments described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other embodiments can be within the scope of the following claims.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

As used herein, the phrase “and/or” as in “A and/or B” refers to A alone, B alone, and A and B.

As used herein, the term “user” can refer to any entity including a person or a computer.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

Furthermore, the skilled artisan will recognize the interchangeability of various features from different embodiments disclosed herein and disclosed in U.S. Pat. No. 63/034,331; U.S. Pat. Nos. 9,008,745; 9,182,368; 10,173,042; U.S. application Ser. No. 15/601,204 (US Pub. No. 2017/0251958); U.S. application Ser. No. 15/876,678 (U.S. Pub. No. 2018/0140235); U.S. application Ser. No. 14/812,288 (US Pub. No. 2016/0029931); U.S. application Ser. No. 14/812,288 (US Pub. No. 2016/0029966); US Pub. No. 2017/0128009; U.S. App. No. 62/855,194; U.S. App. No. 62/854,088; U.S. App. No. 62/970,282; U.S. App. No. 63/188,641; PCT App. No. PCT/US19/49270 (WO2020/051101), U.S. application Ser. No. 17/236,753; PCT App. No. PCT/2021/028445; and U.S. application entitled SYSTEMS FOR ADAPTIVE HEALTHCARE SUPPORT, BEHAVIORAL INTERVENTION, AND ASSOCIATED METHODS, filed Jun. 3, 2021 (Attorney Docket No. 137553.8018.US01), listing Daniel Goldner et al. as inventors, which are all incorporated by reference. For example, methods of detection, sensors, detection elements, biosensors, user devices, etc. can be incorporated into or used with the technology disclosed herein. All of these applications are incorporated herein by reference in their entireties. Similarly, the various features and acts discussed above, as well as other known equivalents for each such feature or act, can be mixed and matched by one of ordinary skill in this art to perform methods in accordance with principles described herein. All of the above cited applications and patents are herein incorporated by reference in their entireties.

From the foregoing, it will be appreciated that specific embodiments of the invention have been described herein for purposes of illustration, but that various modifications may be made without deviating from the scope of the invention. Accordingly, the invention is not limited except as by the appended claims.

1. A computer-implemented method for notifying a user of a recommended self-care mode, the method comprising:
collecting health data of the user;
identifying a health metric based on the health data, wherein the health metric met a threshold health value;
identifying a self-care mode from a plurality of self-care modes by:
analyzing the plurality of self-care modes, wherein each of the plurality of self-care modes has one or more contributing health factors;
determining predictions of the health metric based on the plurality of self-care modes;
identifying contribution measures of the plurality of self-care modes for the predictions;
selecting the self-care mode from the plurality of self-care modes based on the contribution measures; and
outputting a notification for the user including the self-care mode, the prediction, and the one or more contributing health factors.

2. The computer-implemented method of claim 1, wherein the self-care mode is a first self-care mode, the prediction is a first prediction, and the notification is a first notification, further comprising:
collecting additional heath data of the user;
identifying a second self-care mode from the plurality of self-care modes; and
outputting a second notification for the user including a second prediction.

3. The computer-implemented method of claim 1, wherein:
the health data includes one or more of blood pressure data, blood glucose data, heart rate data, food data, activity data, sleep data, weight data, medication data, diagnosis data, or demographics data;
the prediction of the health metric includes a predicted future value or range for the health metric of the user; and
the one or more contributing health factors include one or more of blood pressure, blood glucose, heart rate, food, activity, sleep, weight, medication, diagnosis, or demographics.

4. The computer-implemented method of claim 1, wherein the health data is a first set of health data, further comprising:
determining the user has implemented the self-care mode;
collecting a second set of health data of the user;
identifying a change in the health metric based on the second set of health data; and
dynamically updating the self-care mode based on the change in the health metric.

5. The computer-implemented method of claim 1, wherein the prediction associated with each self-care mode of the plurality of self-care modes is determined using an optimization algorithm.

6. The computer-implemented method of claim 1, wherein selecting the self-care mode comprises selecting the self-care mode with a largest improvement to the health metric of the predictions.

7. The computer-implemented method of claim 1, wherein the notification includes a recommended action for the user related to the one or more contributing health factors.

8. The computer-implemented method of claim 1, wherein:
the self-care mode is a cardiovascular risk self-care mode,
the health data includes a cardiovascular risk score based on systolic blood pressure data, cholesterol levels, blood pressure variability, or combinations thereof;
the prediction is a change of the cardiovascular risk score; and
the one or more contributing health factors include diet, exercise, weight of the user, or combinations thereof.

9. The computer implemented method of claim 1, wherein the self-care mode provides one or more meal plans, exercise programs, and/or weight loss goals.

10. The computer-implemented method of claim 1, wherein:
the self-care mode is a glucose management self-care mode,
the health data includes blood glucose levels;
the prediction is a maximum blood glucose level, minimum blood glucose level, and/or blood glucose level range; and
the one or more contributing health factors includes diet, weight, exercise, or sleep of the user.

11. The computer implemented method of claim 1, wherein the self-care mode provides actions to be performed at specific times.

12. The computer-implemented method of claim 1, wherein:
the self-care mode is a weight management self-care mode,
the health data includes weight, body mass index, age, and/or sex;
the prediction a change in the weight of the user; and
the one or more contributing health factors include diet, exercise, or sleep.

13. A computing system for notifying a user of a recommended self-care mode, the computing system comprising:
one or more processors; and
one or more memories storing instructions that, when executed by the one or more processor, cause the computing system to perform a process comprising:
collecting health data of the user;
identifying a health metric based on the health data, wherein the health metric met a threshold health value;
identifying a self-care mode from a plurality of self-care modes by:
analyzing the plurality of self-care modes, wherein each of the plurality of self-care modes has one or more contributing health factors;
determining predictions of the health metric based on the plurality of self-care modes;
selecting the self-care mode from the plurality of self-care modes based on a prediction associated with the self-care mode; and
outputting a notification for the user including the self-care mode, the prediction, and the one or more contributing health factors.

14. The computing system of claim 13, wherein the self-care mode is a first self-care mode, the prediction is a first prediction, and the notification is a first notification, and wherein the process further comprises:
collecting additional heath data of the user;
identifying a second self-care mode from the plurality of self-care modes; and
outputting a second notification for the user including a second prediction.

15. The computing system of claim 13, wherein:
the health data includes one or more of blood pressure data, blood glucose data, heart rate data, food data, activity data, sleep data, weight data, medication data, diagnosis data, or demographics data;
the prediction of the health metric includes a predicted future value or range for the health metric of the user; and
the one or more contributing health factors include one or more of blood pressure, blood glucose, heart rate, food, activity, sleep, weight, medication, diagnosis, or demographics.

16. The computing system of claim 13, wherein the health data is a first set of health data, and wherein the process further comprises:
determining the user has implemented the self-care mode;
collecting a second set of health data of the user; and
identifying a change in the health metric based on the second set of health data.

17. The computing system of claim 13, wherein the prediction associated with each self-care mode of the plurality of self-care modes is determined using an optimization algorithm.

18. The computing system of claim 13, wherein selecting the self-care mode comprises selecting the self-care mode with a largest improvement to the health metric of the predictions.

19. The computing system of claim 13, wherein the notification includes a recommended action for the user related to the one or more contributing health factors.

20. A computer-readable storage medium having computer executable instructions stored thereon that, when executed by one or more processors, direct the one or more processors to perform a method for notifying a user of a recommended self-care mode, the method comprising:
collecting health data of the user;
identifying a health metric based on the health data, wherein the health metric met a threshold health value;
identifying a self-care mode from a plurality of self-care modes by:
analyzing the plurality of self-care modes, wherein each of the plurality of self-care modes has one or more contributing health factors;
determining predictions of the health metric based on the plurality of self-care modes;
selecting the self-care mode from the plurality of self-care modes based on a prediction associated with the self-care mode; and
outputting a notification for the user including the self-care mode, the prediction, and the one or more contributing health factors.

21. The computer-readable storage medium of claim 20, wherein the self-care mode is a first self-care mode, the prediction is a first prediction, and the notification is a first notification, and wherein the method further comprises:
collecting additional heath data of the user;
identifying a second self-care mode from the plurality of self-care modes; and
outputting a second notification for the user including a second prediction.

22. The computer-readable storage medium of claim 20, wherein:
the health data includes one or more of blood pressure data, blood glucose data, heart rate data, food data, activity data, sleep data, weight data, medication data, diagnosis data, or demographics data;
the prediction of the health metric includes a predicted future value or range for the health metric of the user; and
the one or more contributing health factors include one or more of blood pressure, blood glucose, heart rate, food, activity, sleep, weight, medication, diagnosis, or demographics.

23. The computer-readable storage medium of claim 20, wherein the health data is a first set of health data, and wherein the method further comprises:
determining the user has implemented the self-care mode;
collecting a second set of health data of the user; and
identifying a change in the health metric based on the second set of health data.

24. The computer-readable storage medium of claim 20, wherein the prediction associated with each self-care mode of the plurality of self-care modes is determined using an optimization algorithm.

25. The computer-readable storage medium of claim 20, wherein selecting the self-care mode comprises selecting the self-care mode with a largest improvement to the health metric of the predictions.

26-58. (canceled)
